(19) United States
(12) Patent Application Publication
(10) Pub. No.: US 2008/0249637 A1
(43) Pub. Date: Oct. 9, 2008

(54) PARTIALLY BIODEGRADABLE THERAPEUTIC IMPLANT FOR BONE AND CARTILAGE REPAIR

(75) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(73) Assignee: CINVENTION AG, Wiesbaden (DE)

(21) Appl. No.: 12/098,282

(22) Filed: Apr. 4, 2008

Related U.S. Application Data
(60) Provisional application No. 60/910,456, filed on Apr. 5, 2007.

(51) Int. Cl. A61F 2/02; A61B 17/08; A61F 2/28

(52) U.S. Cl. 623/23.72; 606/151; 623/16.11

(57) ABSTRACT

Exemplary embodiment of the present invention is directed to an at least partially biodegradable implant suitable for implantation into a subject for repairing a bone or cartilage defect, comprising a three-dimensional open-celled framework structure made of a non-particulate first material, the framework structure being embedded in a second, non-particulate material different from said first material, or the open-celled framework structure being substantially completely filled with said second, non-particulate material, wherein at least one of the first material or the second material is at least partially degradable in-vivo. Furthermore, the present invention is directed to a method for repairing a bone or cartilage defect in a living organism, comprising implanting an implant according to the exemplary embodiment of the present invention into the defective bone or cartilage, or replacing the defective bone or cartilage at least partially.

### CROSS-REFERENCE TO RELATED APPLICATION(S)

The present invention claims priority of U.S. provisional application Ser. No. 60/910,456 filed Apr. 5, 2007, the entire disclosure of which is incorporated herein by reference.

### FIELD OF THE PRESENT INVENTION

The exemplary embodiments of the present invention is directed to an at least partially biodegradable implant suitable for implantation into a subject for repairing a bone or cartilage defect, comprising a three-dimensional open-celled framework structure made of a non-particulate first material, the framework structure being embedded in a second, non-particulate material different from said first material, or the open-celled framework structure being substantially completely filled with said second, non-particulate material, wherein at least one of the first material or the second material is at least partially degradable in-vivo. Furthermore, the present invention is directed to a method for repairing a bone or cartilage defect in a living organism, comprising implanting an implant according to the exemplary embodiment of the present invention into the defective bone or cartilage, or replacing the defective bone or cartilage at least partially.

### BACKGROUND INFORMATION

Implants are increasingly used in surgical, orthopedic, dental and other related applications such as tissue engineering. However, the conventional implant technology is focused on improving implants by making them combination products, i.e. combining drugs or therapeutically active agents with implants, such as drug-eluting coatings, or by incorporating those agents into the implant body. Other research and development is focused on increasing the contact surface between the tissue and implant surface. In some specific treatment, bone defects are treated by using cements or cement-like materials comprising ceramic materials or polymer ceramic composites. Also, the treatment of bone defects can involve the implantation of an autograft, an allograft, or a xenograft in the defected site. Biological implants and grafts suffer of many issues such as shortage of donor tissue, infectious contamination by bacteria or virus and others. A synthetic implant may comprise in those cases potential alternatives.

One of the important issues is that due to biomechanical and physiologic requirements an implant material should have a certain mechanical strength or elasticity to be incorporated into the target tissue and anatomic region, on the other hand desired functions such as degradability or incorporating beneficial agents such as pharmacologically or therapeutically active agents are mostly contradictory the foregoing. For example, a range of bone grafting materials are established in clinical use, such as demineralized human bone matrix, bovine collagen mineral composites and processed coralline hydroxyapatite, calcium sulphate scaffolds, bioactive glass scaffolds and calcium phosphate scaffolds. Such orthopedic implants can be used as both temporary and permanent conduits for bone. Those materials may also be used to facilitate and direct the growth of bone or cartilage tissue across sites of fractures or to re-grow them in defective, damaged or infected bone. The provision of appropriate implants also requires considering the structure of bone that has to be treated. Cortical and cancellous bone are structurally different, although the material composition is very similar. Cancellous bone comprises a thin interstitium lattice interconnected by pores of 500-600 micron width with a spongy and open-spaced structure, whereby the interstitium can be substituted by a scaffolding material. Cortical bone comprises neurovascular “Haversian” canals of about 50-100 micron width within a hard or compact interstitium. A scaffold may allow at least osteoconduction or osteoinduction. Osteoinductive materials actively trigger and facilitate bone growth, for example by recruiting and promoting the differentiation of mesenchymal stem cells into osteoblasts. Osteoconductive materials induce bone to grow in areas where it would not normally grow, also called “ectopic” bone growth, usually by biochemical and/or physical processes. Osteogenic materials contain cells that can form bone or can differentiate into osteoblasts.

For example, it can be desirable to have an implant material that allows osseointegration. Known implants either provide a rough surface, usually made from metals such as titanium, titanium alloys, stainless steel or cobalt chromium, or sometimes a porous surface. When using such materials, the osseointegration is typically only a mechanical integration that typically is poor or incomplete. Other reasons of incomplete integration are due to weak bone of the patient, for example due to cancerous diseases or osteoporosis. However, a rough or porous surface is usually applied to dense metal implants, for example by thermal spraying, surface abrasion, pitting, or other methods. Other solutions may provide a coating of hydroxyapatite, that usually is coated onto the surface of such conventional implants. It is a known issue that the adhesion of hydroxyapatite is not very strong and depending on the physiologic fluids present, in case of inflammation for example comprising acidic pH, the loosening of the hydroxyapatite occurs regularly.

Other reasons for implant failure are that dense implants are embedded non-physiologically into the surrounding tissue, inherently with suboptimal biomechanical integration into the part of the body or tissue, for example frequently causing micro fractures or, because of insufficient osseointegration, micro movements. One typical effect of implant failure, regardless of the real cause, is a peri-implantitis, acute, subacute or chronic inflammation that continuously affects or opposes the intended implant function. Specifically in critical implant regions, such as dental implants, the biologic environment and physiologic conditions is a complicating factor with a higher risk of infections due to the microbial, bacterial or fungi flora. Typical effects that may be caused by peri-implantitis are inflammation of mucosa, loss of attached gingival, exposure of a cervical portion of the implant and loss of the surrounding bone and functional implant failures. Even in dental treatments with extraction of a tooth an open wound is caused that might be contaminated by bacteria. A further significant issue is that the absence of the tooth induces spontaneously alveolar bone remodeling with resulting atrophy. Atrophy may subsequently cause more complex complications for reconstruction.

German Application DE 19901271 describes an implant for reconstruction of bone defects comprising a highly pure aluminum oxide and/or zirconium oxide ceramic, the surface of which is at least partially coated with tricalcium phosphate or hydroxylapatite. An independent claim is also included for a method of reconstructing bone defects by inserting the ceramic implant, where an implant (or a mold for casting an implant) corresponding to the image site is prepared using an imaging process and the implant is coated before insertion. U.S. Patent Publication No. 2005/249773 describes a degradable implant composition based on biocompatible ceramics and minerals, biocompatible glasses, and biocompatible polymers, and the use thereof for e.g. in-situ replicating a bone defect, or shaping an implant in a mold ex-situ. European Patent Publication EP 1344538 describes a method to produce and a porous biodegradable implant based on biocompatible ceramics, biocompatible glasses, biocompatible polymers, and combinations thereof. U.S. Pat. No. 5,282,861 describes a bone implant consisting of an open-celled tantalum structure formed by vacuum deposition of a thin tantalum layer onto a reticulated carbon foam, resulting in a lightweight porous structure mimicking the micro-structure of cancellous bone for osteconduction. U.S. Pat. No. 6,087,553 describes an implant obtained by interdigitating polyethylene to a desired depth into the surface of an implant as described in U.S. Pat. No. 5,282,861, to provide a surface of the implant being smooth and having less friction. None of these documents teach or disclose filling the pore system of an open-celled structure with degradable material.

There are several disadvantages related to the use of ceramic materials in implant materials. For example, the main disadvantage of using hydroxyl apatite crystalline forms in such materials is its lack of microporosity and mechanical stability. For adequate bone in-growth it is conventionally known that a porosity of e.g., at least 100 μm or even more is required that cannot be obtained by ceramic or crystalline forms of hydroxyl apatite. Another drawback is the inferior mechanical stability of hydroxylapatite that is brittle and thus typically not suitable for stem replacement in implants. Conventional solutions with only coating a metal implant surface with hydroxyl apatite are prone to fatigue-related destruction of the coating. The application of hydroxyl apatite based cements further comprises a significant issue of mechanical stability and stress shielding as the formation of natural bone tissue is a physiologic process over time whereby during the engraftment phase the materials based on or including hydroxyl apatite do not provide a sufficient biomechanical stability unless the engraftment process is completed. The use of polymers also comprises constraints due to the fact that polymers are prone to suffer from creep and fatigue.

Metallic implant materials are usually favorable in terms of toughness, ductility and fatigue resistance. On the other hand they are known to be stiffer than natural bone, resulting in stress shielding. The phenomenon of stress shielding is well known and based on the effect that the implant material bears more of mechanical loads if it is stiffer than the surrounding tissue. This results in a “shielding” of the natural bone tissue from the mechanical load triggering the resorption processes of bone. Other ceramic implant materials are known to be prone to micro cracks, particularly when impulsive forces occur.

A further known issue is that several implant materials, particularly polymer or ceramic based materials are often hardly detectable by non-invasive imaging methods after implantation.

### SUMMARY OF EXEMPLARY EMBODIMENTS OF PRESENT INVENTION

One exemplary object of the present invention is to provide implants for orthopedic, surgical, dental and traumatologic implants, particularly implants for substituting or repairing, e.g., bone defects.

For example, the implant can be made from materials that may provide an adjustable, accurate biodegradation in-vivo, and may be tailored to provide additional functions, such as incorporating or releasing beneficial agents.

According to an exemplary embodiment of the present invention, an at least partially biodegradable implant is provided which is suitable for implantation into a subject for repairing a bone or cartilage defect. The implant comprises a three-dimensional open-celled framework structure made of a non-particulate first material, the framework structure being embedded in a second, non-particulate material different from said first material, or the open-celled framework structure being substantially completely filled with said second, non-particulate material, wherein at least one of the first material or the second material is at least partially degradable in-vivo. For example, the implant is substantially non-porous, i.e. the pores or openings of the framework structure of the first material are substantially completely filled with the second material to provide a densely packed, substantially non-porous implant.

Such exemplary structure can have osteoinductive or osteoconductive properties, i.e. it may actively trigger and facilitate bone growth, for example by recruiting and promoting the differentiation of mesenchymal stem cells into osteoblasts, it may induce bone to grow in areas where it would not normally grow, also called “ectopic” bone growth. For example, the framework structure may have a bulk volume porosity of about 10-90%. The implant may form a structure having a spongy or trabecular open-spaced lattice structure of interconnected continuous channels built from a first material. In exemplary embodiments, the channels/pores in the first material may have a dimension, e.g. diameter or length, suitable for osteoconduction, such as from about 200 to 1000 μm.

The implant may be used for repairing a bone, tooth or cartilage defect in a living organism by implanting the implant into a subject, such as a human being, in-vivo. Furthermore, the implant may be used to replace natural bone or cartilage in a living organism in-vivo. For example, the implant may be an implantable tissue replacement, an implantable fracture fixation device such as plates, screws and rods, a dental implant, an orthopedic implant, a traumatologic implant, or a surgical implant.

In an exemplary embodiment of the present invention, the non-particulate first or second material includes at least one of a metal or a metal alloy. Furthermore, in exemplary embodiments, any of the materials can be completely degradable in-vivo, but with different rates of degradation if both are selected degradable.

According to an alternative exemplary embodiment of the present invention, the non-particulate first material is substantially not degradable in-vivo, but the second material is degradable, or vice versa, or both materials are degradable. In such embodiments, the in-vivo degradation rates of the matrix material and the non-particulate metallic material is different to provide after implantation, the formation of an osteconductive, porous structure by preferential degradation of the faster degradable material. For example, in certain exemplary embodiments, the in-vivo degradation rate of the second material is lower than the degradation rate of the first material. In other embodiments, the in-vivo degradation rate of the second material is higher than the degradation rate of the non-particulate metallic material.

In an exemplary embodiment, the non-particulate first or second material is selected such that its in-vivo degradation rate substantially matches with the re-growth or repair rate of the natural bone, e.g. the degradation rate of the material may be in a range of from about 3 to 8 weeks. In other exemplary embodiments, the non-particulate first or second material is selected such that its in-vivo degradation rate substantially matches with the regrowth or repair rate of the natural cartilage, e.g. the degradation rate of the material may be in a range of from about 4 to 10 weeks.

According to a further exemplary embodiment of the present invention, the implant can include first or second materials selected from a biocorrosive alloy, or a mixture of at least one first metallic material and at least one second metallic material, the first metallic material being more electronegative than the second metallic material, such that the first and second non-particulate metallic material form a local cell at their contact surfaces. In such an embodiment, the less noble metal is preferentially degraded in-vivo.

In certain exemplary embodiments, the non-particulate first or second, preferable the second material includes an organic material such as a polymer or copolymer, which may be a biodegradable polymer. In other embodiments, the second may itself consist of a metallic material such as a metal or an alloy, or may consist of a ceramic material.

According to a further exemplary embodiment of the present invention, the first or second material can include an inorganic-organic hybrid material, for example a material obtainable by sol-gel processing. Also, the first or second material may include a combination of any of the above described materials.

Also, the implants of exemplary embodiments may further comprise, in the first or second material, conventional additives such as a solvent, a filler, a pigment, or a beneficial agent, which may optionally be configured to be released in-vivo from the implant after insertion into the living organism.

According to a further exemplary embodiments of the present invention, a method for repairing a bone or cartilage defect in a living organism can be provided, comprising implanting an at least partially degradable implant as defined herein into the defective bone or cartilage, or replacing the defective bone or cartilage at least partially with the implant.

Another exemplary embodiments of the present invention can provide a class of implants whereby the mechanical, chemical, biological and physical properties such as electrical conductivity, optical or other suitable properties can be tailored appropriately to the intended use.

Further exemplary embodiments can include the implant as described herein which may comprise rationally designed structures to allow engraftment, ingrowth, induction or conduction or any combination thereof.

### BRIEF DESCRIPTION OF THE DRAWINGS

Further objects, features and advantages of the present invention will become apparent from the following detailed description taken in conjunction with the accompanying Figures showing illustrative embodiments of the present invention, in which:

FIG. 1 is a schematic illustration of an exemplary trabecular structure of a first material of the implant according to an exemplary embodiment of the present invention, e.e., which can mimic natural cancellous or “spongy” bone;

FIG. 2 is a schematic illustration of a part of an implant according to another exemplary embodiment of the present invention, having interconnected spaces/channels within an open-celled matrix of a second material, with the spaces being unfilled, e.g., with the first material not shown therein;

FIG. 3 is a schematic illustration of a part of an implant according to another exemplary embodiment of the present invention, having interconnected spaces/channels within an open-celled second material, with the spaces being unfilled, e.g., with the first material not shown therein; and

FIG. 4 is a schematic diagram of a section of a part of an implant according to still another exemplary embodiment of the present invention, with the spaces being unfilled, e.g., with one of the first or second material not shown therein.

Throughout the Figures, the same reference numerals and characters, unless otherwise stated, are used to denote like features, elements, components or portions of the illustrated embodiments. Moreover, while the subject invention will now be described in detail with reference to the Figures, it is done so in connection with the illustrative embodiments. It is intended that changes and modifications can be made to the described embodiments without departing from the true scope and spirit of the subject invention.

### DETAILED DESCRIPTION OF EXEMPLARY EMBODIMENTS

The terms “active ingredient”, “active agent” or “beneficial agent” can include but not limited to any material or substance which may be used to add a function to the implantable medical device. Examples of such active ingredients can include biologically, therapeutically or pharmacologically active agents such as drugs or medicaments, diagnostic agents such as markers, or absorptive agents. The active ingredients may be a part of the first or second particles, such as incorporated into the implant or being coated on at least a part of the implant. Biologically or therapeutically active agents comprise substances being capable of providing a direct or indirect therapeutic, physiologic and/or pharmacologic effect in a human or animal organism. A therapeutically active agent may include a drug, pro-drug or even a targeting group or a drug comprising a targeting group. An “active ingredient” can include but not limited to a material or substance which may be activated physically, e.g., by radiation, or chemically, e.g., by metabolic processes.

The term “biodegradable” can include but not limited to any biocompatible material which can be removed in-vivo, e.g. by biocorrosion or biodegradation. Thus, any material, e.g., a metal or organic polymer that can be degraded, absorbed, metabolized, or which is resorbable in the human or animal body may be used either for a biodegradable metallic layer or as a biodegradable template in the exemplary embodiments of the present invention. In addition, the terms “biodegradable”, “bioabsorbable”, “resorbable”, and “biocorrodible” can include but not limited to materials that are broken down and may be gradually absorbed or eliminated by the body in-vivo, regardless whether these processes are due to hydrolysis, metabolic processes, bulk or surface erosion.

The term “non-particulate material” can exclude materials having the form of a plurality of particles, thus, for example, the term excludes materials in the form of fibers, spheres, beads etc. as one of the first or second material.

The exemplary present invention is described in greater detail herein with reference to certain exemplary embodiments. The following description makes reference to numerous specific details in order to provide a thorough understanding of certain exemplary embodiments of the present invention. However, each and every specific detail needs not to be employed to practice the exemplary embodiments of the present invention.

According to certain exemplary embodiments of the present, a partially or completely degradable implant for healing of tissue defects can be provided, e.g., such as replacing or repairing bone or cartilage defects in a living organism in need thereof. The exemplary implant may also comprise an orthopedic fixation device such as a rod, screw, nail or plate. Other exemplary embodiments can include to provide an implant in the form of a replica of the defective area for direct replacement of the defective area, such as replacing bone defects induced by surgical craniotomy, or filling tooth roots for dental restoration.

With the implants according to exemplary embodiments of the present invention, being partially or completely degradable in-vivo, an implant is provided, which after implantation can develop into a porous, trabecular structure, which can, e.g., mimic the structure of cancellous or cortical bone, thus providing osteoconductive and/or osteoinductive properties. The implants of the exemplary embodiments of the present invention thus allow to replace natural bone or cartilage material with e.g. an essentially dense and mechanically resilient material directly after implantation. After a certain time in the body, at least a part of the implant can be gradually degraded by degradation of at least one of the first or second material, gradually leaving or releasing a porous, trabecular structure which facilitates or even promotes ingrowth of the natural tissue, thus leading to, e.g., an “anchorage” of at least a part of the implant in the tissue into which it has been implanted. In case of a fully degradable implant, this will be gradually completely replaced over time by re-grown natural tissue.

For example, suitable selection of the non-particulate first material and/or the second material, wherein at least one of these materials is biodegradable, it is possible to provide an implant comprising a biocompatible material that exhibits the desired mechanical properties directly after implantation. Furthermore, it is possible to select the materials used and their combination and structural distribution in the implant such that due to an at least partial degradation of at least one of the materials, e.g. the first particles forming the framework structure, whereby the degradation rate can be controlled, a porous structure is formed in the body which allows a stepwise in-growth of surrounding tissue and an incorporation of the implant material over time, thus promoting healing of the wound or cavity filled. Thus, with the implants of exemplary embodiments of the present invention, a temporarily tailorable variation of the properties of the implant depending on the progress of healing of the defect may be provided.

Thus, before biodegradation starts, the implant allows to mechanically resist biomechanical loads while in the mid- and long-term at least a part of the implant will be replaced during degradation by ingrowing tissue that increases the flexibility and biomechanical properties by substituted natural tissue. Another exemplary advantage is that the exemplary embodiment of the present invention can allow to additionally easily functionalize the implant, for example by incorporating functional compounds such as radiopaque particles such as biocompatible metals, or to tailor specifically the mechanical properties such as flexibility by introducing e.g., fibers. Moreover, the incorporation of, e.g., anti-microbial agents such as silver or copper into the implant can allow to increase the anti-infective properties of the implant.

In another exemplary embodiment, the implant can be inserted into the defective area for replacement of bone or cartilage. For example, the presence of a degradable metallic first material then leaves an open-celled, porous structure consisting of the second material, comprising interconnected channels or pores in the second material matrix by degradation of the metal in-vivo. On the other hand, using a degradable second material will lead to a spongy, trabecular structure of the first material framework left over after degradation of the second material over time. Such structures may promote and/or guide the growth of natural tissue, e.g. bone, so that the implant or at least a part thereof is step by step replaced by the normal, natural tissue. In certain exemplary embodiments, the implant may be designed from completely degradable materials, so that is completely vanishes from the body of the living organism after time, i.e. the implant fulfills only a temporary function.

According to an exemplary embodiment of the present invention, an implant suitable for implantation into a subject for repairing a bone or cartilage defect is provided, the implant comprising a three-dimensional open-celled framework structure made of a non-particulate material, the framework structure being embedded in another non-particulate material, or the open-celled framework structure being substantially completely filled with the other non-particulate material, wherein at least one of the materials is at least partially degradable in-vivo.

While in exemplary embodiments, the implant before implantation may bedense, and the open-celled framework or lattice structure is only developed/laid open by degradation of one of its constituents, e.g. a degradable metallic material, the implant may also have a porous structure, at least partially, before implantation, to facilitate access of physiologic fluids.

In exemplary embodiments, one of the materials can form an open porous structure that has a bulk volume porosity of about 10-90%, preferable from about 30% to 80% and even more preferable from 50% to 80%, and which may be substantially completely filled with the non-particulate other material or embedded therein.

For example, a metallic framework or three-dimensional network or mesh structure as the first material may be embedded in a polymeric matrix material to form the implant, wherein either the matrix or the metallic framework is biodegradable after implantation. If the matrix is degradable, its degradation over time releases the metallic structure step by step, allowing a guided ingrowth of natural tissue into the network structure, which still also serves as a mechanical support. If the metallic framework is degradable, pores and channels in the polymeric matrix are formed in-vivo, into which the natural tissue may grow in a guided manner, step by step replacing the metallic framework.

In another exemplary embodiment, the first material may be a metallic block or plate structure, into which trabecular structures, pores or channels are cut, which may be filled with a polymeric second material or even a different metal to form the implant.

The interconnected channels or pores in the framework structure may define a spongy or trabecular open-spaced lattice structure of interconnecting continuous channels within one of the materials, filled with the second material, which allows tissue ingrowth after removal/degradation of the second material. In an exemplary embodiment for bone repair, the channels/pores are macropores having a dimension suitable for osteoconduction, preferable of about 200 micrometer (μm) to 1000 μm. Pore sizes and porosities may be measured by adsorption methods conventionally used, e.g. N₂ or Hg-adsorption.

In the exemplary embodiments of the present invention, at least one of the materials used can be degradable in-vivo. For example, a first material for the framework structure may be substantially not degradable in-vivo, whereas the non-particulate second material used as a matrix or filling is degradable, or both materials are degradable in-vivo. In one exemplary embodiment, the implant is adapted to provide, after degradation of a first degradable material, an open-celled, interconnected network of channels or pores or capillaries or combined compartments, whereby degradation can take place partially or completely in situ or in-vivo, i.e. in the living body. These compartments are delimited e.g. by the non-degradable or slower degradable second materials that demarcate the interconnected network of hollow channels or pores. In a exemplary embodiment, the first degradable materials are a non-particulate metallic material, and the second material comprises a matrix material such as a polymer or organic-inorganic hybrid material as described herein.

When using degradable materials it can be desirable, that the degradation rate approximately matches to the re-growth or repair rate of the tissue treated. Typical biodegradation rates for maintaining the structure or structural integrity of a scaffold can be for example 4-10 weeks for cartilage repair and 3-8 weeks for bone repair. The mechanical requirements of the implants are highly dependant on the type of tissue being replaced, for example cortical bone has a Young Modulus of 15-30 GPa, whereby cancellous (or spongy, trabecular) bone has a Young Modulus of 0.01-2 GPa. Cartilage has a Young Modulus of less than 0.001 GPa. It is desirable that the materials used for an implant in any particular case should reflect this as far as possible.

Thus, in an exemplary embodiment of the present invention, the combination of materials used for the implant is appropriately selected to provide an implant having a Youngs modulus corresponding to cancellous natural bone, preferable in the range from about 0.01 to about 2 GPa, preferable from about 0.1 to 1 GPa, most preferable from 0.8 to 1 GPa.

In another exemplary embodiment of the present invention, the combination of materials used for the implant is appropriately selected to provide an implant having a Youngs modulus corresponding to cortical natural bone, preferable in the range from about 15 to about 30 GPa, preferably from 18 to 28 GPa, and further preferably from 22 to 27 GPa.

In another exemplary embodiment where both materials are degradable in-vivo, the in-vivo degradation rate of the second material and the first material may be different, e.g., the in-vivo degradation rate of the second material can be lower than the degradation rate of the first material, or vice versa.

In other exemplary embodiments, the non-particulate first or second material may be selected such that the in-vivo degradation rate of the material matches with the re-growth or repair rate of the natural bone, wherein the degradation rate of the non-particulate first or second material is preferable in a range of from about 3 to 8 weeks, more preferably from about 8 to 12 weeks and even more preferably more than 3 months.

In other exemplary embodiments, the non-particulate first or second material may be selected such that the in-vivo degradation rate of the material matches with the regrowth or repair rate of the natural cartilage, wherein the degradation rate of the non-particulate first or second material is preferable in a range of from about 4 to 10 weeks, more preferable from 8 to 12 weeks and most preferable more than 3 months.

Exemplary Metallic Materials

In an exemplary embodiment of the present invention, the non-particulate first or second material includes at least one of a metal or a metal alloy, e.g. selected from main group metals of the periodic system, transition metals such as copper, gold, silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or from rare earth metals, and alloys or any mixtures thereof.

The non-particulate first or second material used in some exemplary embodiments can be, without excluding others, selected from, e.g.,—iron, cobalt, nickel, manganese or mixtures thereof, e.g. iron-platinum-mixtures, or as an example for magnetic metal oxides iron oxides and ferrites. Particularly for exemplary implants with magnetic or signaling properties in general, magnetic metals or alloys such as ferrites, e.g. gamma-iron oxide, magnetite or ferrites of Co, Ni, Mn may be used. Examples are described in International Patent Publication Nos. WO83/03920, WO83/01738, WO85/02772, WO89/03675, WO88/00060, WO90/01295 and in WO90/01899 and in U.S. Pat. Nos. 4,452,773, 4,675,173 and 4,770,183, in. In certain exemplary embodiments, it can be preferable to select the non-particulate metallic material from shape memory alloys such as nickel titanium, nitinol, copper-zinc-aluminum, copper-aluminum-nickel, and the like.

In other exemplary embodiments, the non-particulate first or second material are selected from biodegradable metals or alloys, or metal composites. Suitable biodegradable metals can include, e.g., metals, or metal alloys, including alkaline or alkaline earth metals, Fe, Zn or Al, such as Mg, Fe or Zn, and optionally alloyed with or combined with other particles selected from Mn, Co, Ni, Cr, Cu, Cd, Pb, Sn, Th, Zr, Ag, Au, Pd, Pt, Si, Ca, Li, Al, Zn and/or Fe.

In addition, metal oxides, nitrides carbides, ceramic materials etc. may be added in certain exemplary embodiments, e.g., alkaline earth metal oxides or hydroxides such as magnesium oxide, magnesium hydroxide, calcium oxide, and calcium hydroxide or mixtures thereof.

In exemplary embodiments, the non-particulate metallic material may be selected from biodegradable or biocorrosive metals or alloys based on at least one of magnesium or zinc, or an alloy comprising at least one of Mg, Ca, Fe, Zn, Al, W, Ln, Si, or Y, such as e.g. a Mg—Ca alloy, Mg—Zn alloy, Mg-AI—Zn alloy, e.g. commercially available AZ91D, LAE442, AE21.

Furthermore, the non-particulate first or second material may be substantially completely or at least partially degradable in-vivo. Examples for suitable biodegradable alloys comprise e.g. magnesium alloys comprising more than 90% of Mg, about 4-5% of Y, and about 1.5-4% of other rare earth metals such as neodymium and optionally minor amounts of Zr, wherein the components are selected to add up to 100%.; or biocorrosive alloys comprising as a major component tungsten, rhenium, osmium or molybdenum, for example alloyed with cerium, an actinide, iron, tantalum, platinum, gold, gadolinium, yttrium or scandium.

In a further exemplary embodiment of the present invention, the degradable non-particulate first or second material may comprise a metal alloy of (i) 10-98 wt.-%, such as 35-75 wt.-% of Mg, and 0-70 wt.-%, such as 30-40% of Li and 0-12 wt.-% of other metals, or (ii) 60-99 wt.-% of Fe, 0.05-6 wt.-% Cr, 0.05-7 wt.-% Ni and up to 10 wt.-% of other metals; or (iii) 60-96 wt.-% Fe, 1-10 wt.-% Cr, 0.05-3 wt.-% Ni and 0-15 wt.-% of other metals, wherein the individual weight ranges are selected to add up to 100 wt.-% in total for each alloy.

Preferable, the non-particulate first or second material includes one of Mg, Zn, Ca, whereby the metallic material forms upon its degradation in-vivo a substance that has osteoinductive properties.

In such embodiments, the non-particulate first or second material may be degraded to produce e.g. hydroxyl apatite and hydrogen within the living body in the presence of physiologic fluids. Hydroxyl apatite may induce or guide ingrowths of natural surrounding tissue into the residual implant structure. This property of the exemplary implant material is especially advantageous for implants with a temporary function, but with sufficient mechanical stability compared to bioceramics or hydroxyl apatite or polymers alone.

For example, in a first step, a substantially dense implant is inserted, which is capable to immediately fulfill its functions, e.g. to provide mechanical support. Subsequently, during a period of several days, weeks or months, depending on the use of the implant, a part of the implant, e.g. the metallic material, is degraded, leaving behind an open porous network structure of the other material.

Whereas the network structure of channels or spaces formed may have an osteoconductive function during ingrowth of surrounding tissue, the degradation products of the degradable metallic materials may additionally have osteoinductive properties, e.g. promoting the formation of new tissue. Overall, with the material composition and the structure of the implants of exemplary embodiments of the present invention, faster healing and/or better ingrowth of tissue or even complete replacement of the implant by natural tissue may be provided.

According to exemplary embodiments of the present invention, by alloying the aforesaid non-particulate metallic materials it is e.g. possible to tune the physiologic degradation rate from a few days up to 20 years. Moreover, by introducing precious metals either within the alloy of one of the materials, or by combining a first metallic material with a second, less noble metallic material as the second material, or alternatively by applying a currency for example with an appropriate electrode or similar device, the degradation of the less noble metallic material can be substantially altered. Using a metal also allows to utilize the mechanical strength of these compounds and to realize tailored implants that both address the mechanical requirements e.g. immediately after implantation for supportive functions, as well as the biodegradability for later provision or facilitation of tissue ingrowth and incorporation of the residual implant material, if any, into the bone or other tissue.

For example, the implant composition of the exemplary embodiments of the present invention can rationally be tailored by suitably adjusting the metal composition to induce a controlled corrosion. Corrosion occurs when two metals, with different potentials, are in electrical contact while immersed or at least in contact in an electrically conducting corrosive liquid, such as physiologic fluids. Because the metals have different natural potentials in the liquid, a current will flow from the anodic (more electronegative) metal to the cathodic (more electropositive) metal, which will increase the corrosion of the anode. This additional corrosion is also called bimetallic corrosion. It is also preferable to as a galvanic corrosion, dissimilar metal corrosion or contact corrosion. In general, the degradation reactions which occur are similar to those that would occur on a single, uncoupled metal, but the rate of attack is increased, sometimes dramatically. With some metal combinations the change in the electrode potential in the couple potential can induce corrosion which would not have occurred in the uncoupled state (e.g. pitting). The effect of coupling the two metals together can increase the corrosion rate of the anode and reduces or even suppresses corrosion of the cathode. Mostly, bimetallic corrosion occurs in solutions containing dissolved oxygen, and in most neutral and alkaline liquids the primary cathodic reaction is the reduction of dissolved oxygen, while in acidic liquids the cathodic reaction is often the reduction of hydrogen ions to hydrogen gas. Under uncoupled corrosion the anodic and cathodic reactions occur at small, local areas on the metal. In a bimetallic couple the cathodic reaction is more, or totally, on the electropositive member of the couple and the anodic reaction is mostly, or totally, on the electronegative component of the couple.

Using these principles, the corrosion applied to a non-particulate metallic material in the implants of embodiments of the present invention can be a rationally tailored corrosion that can be verified by selecting suitable non-particulate metallic materials and/or combinations thereof with regard to their electronegativity or electropositivity.

Concerning the corrosion control with regard to a non-particulate metallic material, basically two approaches toward implant design may be used. The first is the combination of a first metal or metal alloy with identical or similar electronegativity together with at least one second entity of metal or metal alloy with a different electronegativity that is sufficient to affect the corrosion rate of the first material. The second basic approach is based on selecting more electronegative non-particulate metallic materials that are included in a matrix comprising a different metal that is more electropositive, or vice versa. However, any combination of the foregoing approaches may also be used according to the exemplary embodiment of the present invention.

According to an exemplary embodiment of the present invention, the non-particulate first and second materials comprise at least one first metallic material and at least one second metallic material, the first metallic material being more electronegative than the second metallic material, such that the first and second non-particulate metallic material form a local cell at their contact surfaces. In such an embodiment, the less noble metal is preferentially degraded in-vivo.

In an exemplary embodiment of the present invention, the first material is selected from one of the metallic materials as described above and embedded in or filled with a second, non-metallic, non-particulate matrix material as described below.

In an alternative exemplary embodiment of the present invention, the second material is selected from one of the metallic materials as described above and the first material forming the framework is selected from a non-metallic, non-particulate matrix material as described below.

In a further alternative exemplary embodiment of the present invention, the first and the second material are selected from one of the metallic materials as described above, however from different metallic materials.

In a still further exemplary embodiment of the present invention, the first and the second material are selected from one of the non-metallic, non-particulate matrix materials as described below, however different materials.

Organic Material

According to an exemplary embodiment, the implant as described herein can include an organic material as the first or second non-particulate material.

For example, the organic material may comprise an oligomer, polymer or copolymer such as a poly(meth)acrylate, unsaturated polyester, saturated polyester, polyolefines, polyethylene, polypropylene, polybutylene, alkyd resins, epoxy-polymers or resins, polyamide, polyimide, polyetherimide, polyamideimide, polyesterimide, polyester amide imide, polyurethane, polycarboxylate, polycarbonate, polystyrene, polyphenol, polyvinyl ester, polysilicone, polyacetal, cellulosic acetate, polyvinylchloride, polyvinyl acetate, polyvinyl alcohol, polysulfone, polyphenylsulfone, polyethersulfone, polyketone, polyetherketone, polybenzimidazole, polybenzoxazole, polybenzthiazole, polyfluorocarbons, polyphenylene ether, polyarylate, or cyanatoester-polymers, and any of the copolymers and any mixtures thereof.

One exemplary option is to use a biocompatible, but non-degradable polymer, such as polymethylmethacrylate and/or other acrylic co-polymers, preferable acrylic-terminated butadiene-styrene block copolymers, or cyanoacrylates, polyetherketone or polyetheretherketone, pre-polymers or any mixture thereof. Alternatively, a biodegradable polymer may be used.

According to an exemplary embodiment of the present invention, the organic material comprises a biocompatible and/or biodegradable polymer or copolymer such as collagen, albumin, gelatin, hyaluronic acid, starch, cellulose, methylcellulose, hydroxypropylcellulose, hydroxypropylmethyl-cellulose, carboxymethylcellulose-phthalate; gelatin, casein, dextrane, polysaccharide, fibrinogen, poly(D,L lactide), poly(D,L-lactide-co-glycolide), poly(glycolide-co-trimethylene carbonates), poly(glycolide), poly(hydroxybutylate), poly(allylcarbonate), poly(a-hydroxyesters), poly(ether esters), poly(orthoester), polyester, poly(hydroxyvaleric acid), polydioxanone, poly(ethylene terephthalate), poly(maleic acid), poly(malic acid), poly(tartaric acid), polyanhydride, polyphosphazene, poly(amino acids), polypeptides, polycaprolactones, poly(propylene fumarates), poly(ester amides), poly(ethylene fumarates), poly(hydroxy butyrates), and polyurethanes, or mixtures thereof. In such embodiments, the organic material may be selected from partially or substantially completely biodegradable polymers.

Further polymers which may be used include, for example, poly(meth)acrylate, unsaturated polyester, saturated polyester, polyolefines such as polyethylene, polypropylene, polybutylene, alkyd resins, epoxy-polymers or resins, polyamide, polyimide, polyetherimide, polyamideimide, polyesteramide, polyester amide imide, polyurethane, polycarbonate, polystyrene, polyphenol, polyvinyl ester, polysilicone, polyacetal, cellulosic acetate, polyvinylchloride, polyvinyl acetate, polyvinyl alcohol, polysulfone, polyphenylsulfone, polyethersulfone, polyketone, polyetherketone, polybenzimidazole, polybenzoxazole, polybenzothiazole, polyfluorocarbons, polyphenylene ether, polyarylate, cyanatoester-polymers, and mixtures or copolymers of any of the foregoing.

In certain exemplary embodiments, the polymer material can be selected from poly(meth)acrylates based on mono(meth)acrylate, di(meth)acrylate, tri(meth)acrylate, tetra-acrylate and penta-acrylate monomers; as well as mixtures, copolymers and combinations of any of the foregoing, wherein the metallic particles may be included already during polymerization.

For example, the first or second material may be a polymerization product of a monofunctional monomer such as at least one of methyl acrylate, methyl methacrylate, ethyl acrylate, ethyl methacrylate, butyl acrylate, butyl methacrylate, acryl acrylate, acryl methacrylate, hydroxyethyl acrylate, hydroxyethyl methacrylate, methoxyethyl acrylate, and methoxyethyl methacrylate; or a polymerization product of a polyfunctional monomer which may include at least one of bifunctional aliphatic acrylates, bifunctional aliphatic methacrylates, bifunctional aromatic acrylates, bifunctional aromatic methacrylates, trifunctional aliphatic acrylates, trifunctional aliphatic methacrylates, tetrafunctional acrylates, and tetrafunctional methacrylates, such as triethylene glycol diacrylate, triethylene glycol dimethacrylate, 2,2-bis(4-methacryloxyphenyl)propane, 2,2-bis(4-methacryloxyethoxyphenyl)propane, 2,2-bis(4-methacryloxypolyethoxyphenyl]propane, 2,2-bis[4-(3-methacryloxy-2-hydroxypropoxy)-phenyl]propane, di(methacryloxyethyl) trimethylhexamethylene diurethane, tetramethylolmethane tetraacrylate, and tetramethylolmethane tetramethacrylate, or a di(meth)acrylate, such as urethane dimethacrylate, ethyleneglycol dimethacrylate, (2,2-bis[4-(2-hydroxy-3-methacryloxypropoxy)phenyl]propane (BIS-GMA), (2,2-bis[4-(methacryloxy)phenyl]propane (BIS-MA), 2,2-bis(4-methacryloxyphenyl)propane, 1,2-butanediol-diacrylate, 1,4-butanediol-diacrylate, 1,4-butanediol-dimethacrylate, 1,4-cyclo-hexanediol-dimethacrylate, 1,10-decanediol-dimethacrylate, diethylene-glycol-diacrylate, dipropyleneglycol-diacrylate, dimethylpropanediol-dimethacrylate, triethyleneglycol-dimethacrylate (TEGDMA), tetraethyleneglycol-dimethacrylate, 1,6-hexanediol-diacrylate, 1,6-bis-[2-methacryloxyethoxycarbonylamino]-2,2,4-trimethylhexane (UDMA), neopentylglycol-diacrylate, polyethyleneglycol-dimethacrylate, tripropyleneglycol-diacrylate, 2,2-bis-[4-(2-acryloxyethoxy)phenyl]-propane, 2,2-bis-[4-(2-hydroxy-3-methacryloxypropoxy)phenyl]propane, bis(2-methacryloxyethyl)N,N-1,9-nonylene-biscarbamate, 1,4-cyclohexanedimethanol-dimethacrylate, and diacrylic urethane oligomers. Also, mixtures of monofunctional and polyfunctional monomers may be used.

Exemplary Sol-Gel-Systems

According to a further exemplary embodiment of the present invention, the first or second material can include an inorganic-organic hybrid material, for example a material obtainable by conventional sol-gel processing or combined sol-gel-processing and polymerization reactions. The sol-gel processing can be either a hydrolytic or non-hydrolytic sol-gel processing, for example by using sol-gel forming materials including at least one metal alkoxide.

For example, the metal alkoxide can be selected from at least one of silicon alkoxides, tetraalkoxysilanes, oligomeric forms of tetraalkoxysilanes, alkylalkoxysilanes, aryltrialkoxysilanes, (meth)acrylsilanes, phenylsilanes, oligomeric silanes, polymeric silanes, epoxysilanes; fluoroalkylsilanes, fluoroalkyltrimethoxysilanes, or fluoroalkyltriethoxysilanes, optionally further comprising at least one crosslinking agent including at least one of isocyanates, silanes, (meth)acrylates, 2-hydroxyethyl methacrylate, propyltrimethoxysilane, 3-(trimethylsilyl)propyl methacrylate, isophorone diisocyanate, hexamethylenediisocyanate (HMDI), diethylenetriaminoisocyanate, 1,6-diisocyanatohexane, or glycerin.

In an exemplary embodiment, the first or second material may be obtained from a reaction mixture comprising a metal alkoxide including a hydrolytically condensable, organically modified trialkoxysilane which contains free-radically polymerizable acrylate or methacrylate groups or cyclic groups capable of ring opening polymerization. Examples include, e.g. those based on polysilicid acid modified with polymerizable alkoxy groups or cyclic siloxanes and a mixture of Bis-GMA and 2-hydroxyethyl methacrylate (HEMA). These materials can be cured by hydrolysis and condensation with simultaneous radical polymerization of the resultant alcohols. Functionalized trialkoxysilanes of the R—Si(OR′)₃ type may also be used (with e.g. R and/or R′ representing C₁ to C₂₀ alkyl, alkenyl or alkinyl, wherein R can include at least one acrylic or methacrylic acid functionality), which can condensate, resulting in polysilsesquioxanes RSiO_{3/2}, or which can be co-condensated with other alkoxysilanes or metal alkoxides. Methacrylates may also be used in combination with e.g. tetraethylorthosilicate (TEOS) to provide PMMA-silica hybrids as the matrix material after curing by polymerization and co-condensation.

An overview on several of these precursors for inorganic-organic hybrid materials suitable for the matrix material of the present invention is disclosed in N. Moszner and S. Klapdohr, Nanotechnology for dental composites, Int. J. of Nanotechnology, vol. 1, No. ½, 2004, 130-156, and the references cited therein. All materials preferable to and mentioned therein are in principle also suitable for producing the matrix material in the implants according to certain exemplary embodiments of the present invention. For example, hydrolysable and condensable trialkoxysilanes bearing methacrylate groups can be used, which are connected to the Si-atom via spacers, and silanediacrylates can be preferable materials which can be hydrolyzed and condensated into fluid sols, and cured by e.g. visible light by polymerization of the methacrylate functions.

The precursor compounds of an inorganic-organic hybrid material processable by sol-gel processing may be conventional sol/gel-forming components. The sol/gel-forming components are typically provided in the form of a sol which may comprise a solvent, and which can be cured or hardened by condensation into a gel such as an aerogel or xerogel.

In these embodiments degradable and non degradable metallic particles selected as described above can be combined and mixed with the sol/gel-forming components, or specifically only degradable or non-degradable particles can be used. Optionally, the gel obtained after curing is dissolvable in physiologic fluids, or porous.

In some embodiments, the sol/gel forming components can include metal oxides, metal carbides, metal nitrides, metal oxynitrides, metal carbonitrides, metal oxycarbides, metal oxynitrides, and metal oxycarbonitrides of the above mentioned metals, or any combinations thereof. These compounds, preferable as colloidal particles, can be reacted with oxygen-containing compounds, e.g. alkoxides to form a sol/gel.

If the first or second material comprises a material obtainable by sol-gel processing, substantially all materials and processes as described in applicants WO 2006/077256 may be used.

In an exemplary embodiment, the first or second material may include a combination of any of the above described embodiments of the present invention. For example, hydrolytically condensable metal alkoxides used in sol-gel processing may include at least one polymerizable monofunctional or polyfunctional organic residue, which can be additionally or subsequently subjected to polymerization to produce the first or second material, and such materials may be combined with polymers or the like.

Also, the implants of exemplary embodiments may further comprise conventional additives such as a filler, e.g. salts, hydroxyl apatite; a pigment, or a beneficial agent as further described herein below, which may optionally be configured to be released in-vivo from the final implant

According to an exemplary embodiment of the present invention, the first material may comprise at least about 5 wt.-%, preferable from about 1 to 99 wt.-%, more preferable 10 to 80 wt.-%, most preferable 40 to 75 wt-% of the implants constituents. Furthermore, a non-particulate metallic material can be modified with a coupling agent, preferable a silane coupling agent such as vinyl trichlorosilane, vinyl triethoxysilane, vinyl trimethoxysilane, vinyl tris(beta-methoxyethoxy)silane, and gamma-methacryloxypropyl trimethoxysilane, to improve adherence in the second material or to covalently bond the first material to the second material.

In other embodiments, the first or second material may consist of a metallic material such as a metal or an alloy, or may consist of a ceramic material. Suitable such materials include all biocompatible metals and alloys as well as ceramic materials, including those as described above as materials for the metallic material.

According to the exemplary embodiment of the present invention, the implant after implantation facilitates and enables the formation and organization of tissue, preferable osteoinduction, osteoconduction and formation of natural bone minerals “guided” by the implant fine-structure.

Exemplary Manufacturing

The manufacture of the implant may be done by any suitable conventional manufacturing method. Appropriate techniques include molding a suitable precursor composition in a mold or replica form of the defect to be repaired with the desired design. Also, for example an injection molding processes can be applied. Other exemplary methods include compression molding, compacting, dry pressing, cold isostatic pressing, hot pressing, uniaxial or biaxial pressing, extrusion molding, gel casting, slip casting and tape casting and the like.

The implant must not be necessarily non-porous before implantation or use. However, typically, and preferable, the implant itself, before use/implantation, is non-porous, and for example, porosity can only be created in-vivo by at least partial degradation of at least one of the materials constituting the implant. It can be made of densely welded parts. A metallic material as one of the first or second material may also comprise welded or sintered particles such as sintered pearls, selected and combined as described before, forming a 3-dimensional network structure embedded in a matrix of a non-particulate second material.

In certain exemplary embodiments it is preferable to have a rationally designed distribution of the first material within the implant body, e.g. in the form of a trabecular, spongy framework structure capable to guide tissue growth along pathways released over time by degradation of a part of the implant.

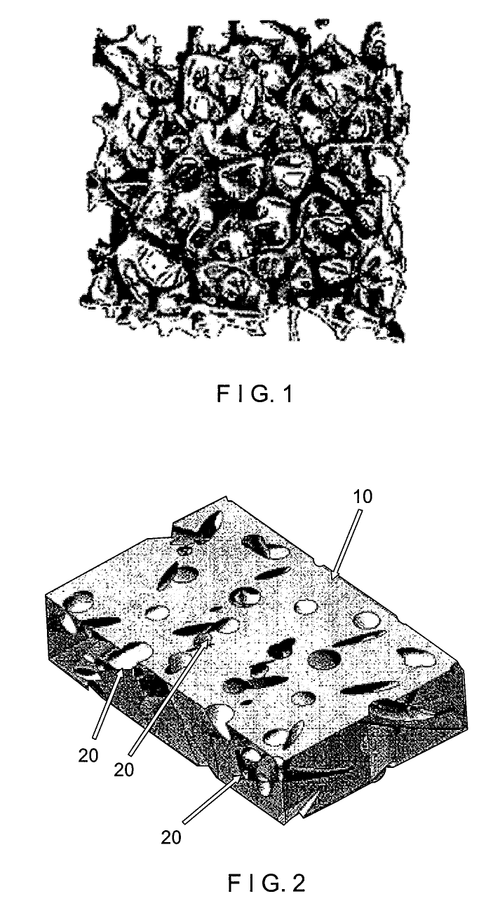

For example, FIG. 1 shows an exemplary trabecular, spongy structure of a part of an implant according to the exemplary embodiment of the present invention, similar to natural cancellous bone. In one exemplary embodiment, the structure shown in FIG. 1 represents the framework of a first material embedded in a non-particulate second material (not shown).

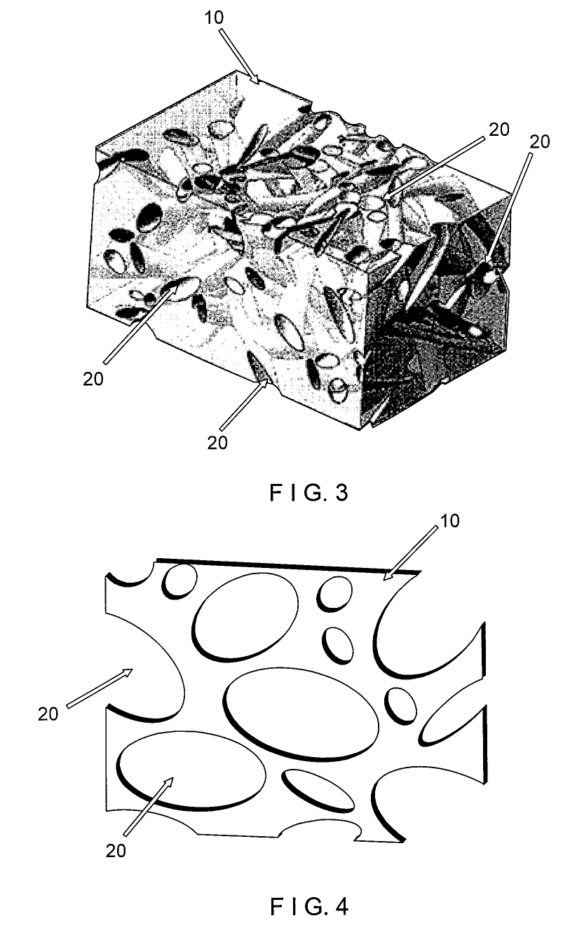

Alternative exemplary embodiments of the implant structure of the present invention, are shown in FIGS. 2 to 4. For example, an open-celled matrix 10 of a second material, for example a polymer, is shown, having a plurality of interconnected spaces or channels 20 extending from the surface of the matrix through its interior, forming a network structure or framework of channels 20. After filling the space/channels 20 with the first material, for example a degradable metal, a substantially dense implant structure is obtained, which after implantation and degradation of e.g. the metallic material provides a hollow structure in the matrix which guides the ingrowth of surrounding natural tissue.

For example, the matrix may also have a structure as described in U.S. Pat. No. 5,282,861, e.g. an open porous polymeric foam or a material derived there from, the pores or spaces thereof being filled with a second material as described herein, wherein at least one of the materials used is biodegradable.

Exemplary manufacturing can be done by various conventional methods. The exemplary implants can be manufactured in one seamless part or with seams out of multiple parts. The present invention, also contemplates the use of different materials for different sections or parts of the exemplary implant. The exemplary implants may be manufactured using conventional implant manufacturing techniques. Particularly, appropriate manufacturing methods can include, but are not limited to, laser cutting, chemical etching or stamping of tubes, for example of the open-celled framework, and then filling the pore system with a liquefied second material. Another option is the manufacturing by laser cutting, chemically etching, and stamping flat sheets, rolling of the sheets and, as a further option, welding the sheets. Other appropriate manufacturing techniques include electrode discharge machining or molding the exemplary implant with the desired design. A further option is to weld or glue individual sections together. Any other suitable implant manufacturing process may also be applied and used. For example, for degradably alloyed implants conventional welding methods are appropriate, or it is possible to structure them, for example introducing open-cellular pores, by foaming or similar methods. Other suitable methods are compression molding, compacting, dry pressing, cold isostatic pressing, hot pressing, uniaxial or biaxial pressing, extrusion molding, gel casting, slip casting and tape casting and the like. A preferable method may be coextruding e.g. strands of the non-particulate metallic material with organic matrix materials, or preparing an open-celled matrix by foaming and subsequently filling the channels/pores with non-particulate metallic material.

In exemplary embodiments, the implant may be shaped as desired, in the form of tubes or sheets or foils or meshes or the like, and then manufactured or welded to the final implant material and/or implant design. Preferable, the parts used comprise different metals, metal oxides or metal alloys. In one exemplary embodiment sheets of e.g. a second matrix material are cut to comprise a porous pattern, mesh-like pattern, trabecular pattern, random or pseudo-random structure or any mixture thereof. They can be stacked together in a sandwich like manner to provide a three dimensional interconnected network of channels, pore or capillaries or combined compartments, serving as the matrix, which is then filled with the first material. Those sheets can be processed to different geometric forms, but however, the sheets can be welded or bonded together to a compact material, for example layer by layer. Preferable, those sheets or foils provide a degradable material themselves, but in certain exemplary embodiments it can be preferable to use different materials in different layers to control corrosion and degradation of specific structural parts of the implant. For example, in certain exemplary embodiments it can be preferable to have alternating layers of a degradable metal or metal alloy and non-degradable metal or metal alloys, if the matrix material is a metallic material itself. In other certain exemplary embodiments, it may be preferable to have alternating layers of a faster degradable material, e.g. a metal alloy or polymer and slower degradable materials, e.g. metal alloys or polymers.

In further exemplary embodiments, the preformed open-celled framework structure is manufactured as the ex-situ form previously, before filling with the non-particulate second material. The channels or pores are then filled up with single or mixed entities of the non-particulate second material. Additionally, other materials such as metals, metal oxides, metal alloys, ceramics, organics, polymers or composites or any mixture thereof, may simultaneously be added during filling of the channels/pores.

Also, a non-particulate first material, for example a metallic material, may be preformed in the form of a network, mesh or woven material of strands or fibers, sintered together, and subsequently embedded in a second material, for example a polymer, which has been melted or dissolved before, followed by hardening.

For example, in a further typical exemplary procedure, the open-celled framework is made from a metallic material by conventional methods as described above, such as manufacturing of porous metal implants by sintering of green bodies, bonding of metal sheets that are perforated by direct laser machining, abrasive water jet machining, stamping, e.g., computer numerical controlled (CNC) stamping, drilling, punching, ion beam or electrochemical or photochemical etching, electrical discharge machining (EDM), or other perforation techniques and/or combinations thereof. The porous framework can then be filled with a polymeric second material, for example a molten or dissolved polymer by conventional methods such as, for example, impregnation, infiltration, dipping, spraying, and the like, to obtain a substantially non-porous, dense implant, wherein the pores in the first material are substantially completely filled with the second material. The basic design of the implants of the exemplary embodiments of the present invention contemplates, that degradation and preferable formation of degradation products such as hydroxyl apatite or the in-growth and engraftment is “guided” as aforesaid.

All exemplary embodiments can comprise both the lattice or framework structure and a degradable matrix structure as well as a non-degradable matrix in any desired three-dimensional orientation or shape.

Exemplary Functionalization

According to the exemplary embodiment of the present invention, additional functions may be provided in the implant by incorporating beneficial agents into at least a part of the implant structure, as desired. Beneficial agents can be selected from biologically active agents, pharmacological active agents, therapeutically active agents, diagnostic agents or absorptive agents or any mixture thereof. Furthermore, the implant may optionally be coated with beneficial agents partially or completely.

Biologically, therapeutically or pharmaceutically active agents according to the exemplary embodiment of the present invention may include a drug, pro-drug or even a targeting group or a drug comprising a targeting group. The active agents may be in crystalline, polymorphous or amorphous form or any combination thereof in order to be used in the present invention. Suitable therapeutically active agents may be selected from the group of enzyme inhibitors, hormones, cytokines, growth factors, receptor ligands, antibodies, antigens, ion binding agents such as crown ethers and chelating compounds, substantial complementary nucleic acids, nucleic acid binding proteins including transcriptions factors, toxines and the like.

Examples of active agents are, for example, cytokines such as erythropoietine (EPO), thrombopoietine (TPO), interleukines (including IL-1 to IL-17), insulin, insulin-like growth factors (including IGF-1 and IGF-2), epidermal growth factor (EGF), transforming growth factors (including TGF-alpha and TGF-beta), human growth hormone, transferrine, low density lipoproteins, high density lipoproteins, leptine, VEGF, PDGF, ciliary neurotrophic factor, prolactine, adrenocorticotropic hormone (ACTH), calcitonin, human chorionic gonadotropin, cortisol, estradiol, follicle stimulating hormone (FSH), thyroid-stimulating hormone (TSH), leutinizing hormone (LH), progesterone, testosterone, toxines including ricine and further active agents such as those included in Physician's Desk Reference, 58th Edition, Medical Economics Data Production Company, Montvale, N.J., 2004 and the Merck Index, 13th Edition (particularly pages Ther-1 to Ther-29), all of which are incorporated herein by reference.

In an exemplary embodiment, the therapeutically active agent is selected from the group of drugs for the therapy of oncological diseases and cellular or tissue alterations. Suitable therapeutic agents are, e.g., antineoplastic agents, including alkylating agents such as alkyl sulfonates, e.g., busulfan, improsulfan, piposulfane, aziridines such as benzodepa, carboquone, meturedepa, uredepa; ethyleneimine and methylmelamines such as altretamine, triethylene melamine, triethylene phosphoramide, triethylene thiophosphoramide, trimethylolomelamine; so-called nitrogen mustards such as chlorambucil, chlornaphazine, cyclophosphamide, estramustine, ifosfamide, mechlorethamine, mechlorethaminoxide hydrochloride, melphalan, novembichin, phenesterine, prednimustine, trofosfamide, uracil mustard; nitroso urea-compounds such as carmustine, chlorozotocin, fotemustine, lomustine, nimustine, ranimustine; dacarbazine, mannomustine, mitobronitol, mitolactol; pipobroman; doxorubicin and cis-platinum and its derivatives, and the like, combinations and/or derivatives of any of the foregoing.

In a further exemplary embodiment, the therapeutically active agent is selected from the group of anti-viral and anti-bacterial agents such as aclacinomycin, actinomycin, anthramycin, azaserine, bleomycin, cuctinomycin, carubicin, carzinophilin, chromomycins, ductinomycin, daunorubicin, 6-diazo-5-oxn-1-norieucin, doxorubicin, epirubicin, mitomycins, mycophenolsaure, mogalumycin, olivomycin, peplomycin, plicamycin, porfiromycin, puromycin, streptonigrin, streptozocin, tubercidin, ubenimex, zinostatin, zorubicin, aminoglycosides or polyenes or macrolide-antibiotics, and the like, combinations and/or derivatives of any of the foregoing.

In a further exemplary embodiment, the therapeutically active agent is selected from the group of radio-sensitizer drugs.

In a further exemplary embodiment, the therapeutically active agent is selected from the group of steroidal or non-steroidal anti-inflammatory drugs.

In a further exemplary embodiment, the therapeutically active agent is selected from agents referring to angiogenesis, such as e.g. endostatin, angiostatin, interferones, platelet factor 4 (PF4), thrombospondin, transforming growth factor beta, tissue inhibitors of the metalloproteinases-1, -2 and -3 (TIMP-1, -2 and -3), TNP-470, marimastat, neovastat, BMS-275291, COL-3, AG3340, thalidomide, squalamine, combrestastatin, SU5416, SU6668, IFN-[alpha], EMD121974, CAI, IL-12 and IM862 and the like, combinations and/or derivatives of any of the foregoing.

In a further exemplary embodiment, the therapeutically-active agent is selected from the group of nucleic acids, wherein the term nucleic acids also comprises oligonucleotides wherein at least two nucleotides are covalently linked to each other, for example in order to provide gene therapeutic or antisense effects. Nucleic acids preferable comprise phosphodiester bonds, which also comprise those which are analogues having different backbones. Analogues may also contain backbones such as, for example, phosphoramide (Beaucage et al., Tetrahedron 49(10):1925 (1993) and the references cited therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)); phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidit-compounds (see Eckstein, Oligonucleotides and Analogues: A Practical Approach, Oxford University Press), and peptide-nucleic acid-backbones and their compounds (see Egholm, J. Am. Chem. Soc. 114:1895 (1992); Meier et al., Chem. Int. Ed. Engl: 31:1008 (1992); Nielsen, Nature, 365:566 (1993); Carlsson et al., Nature 380:207 (1996), wherein these references are incorporated by reference herein. Further analogues are those having ionic backbones, see Denpcy et al., Proc. Natl. Acad. Sci. USA 92:6097 (1995), or non-ionic backbones, see U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996), and non-ribose-backbones, including those which are described in U.S. Pat. Nos. 5,235,033 and 5,034,506, and in chapters 6 and 7 of ASC Symposium Series 580, “Carbohydrate Modifications in Antisense Research”, Ed. Y. S. Sanghui and P. Dan Cook. The nucleic acids having one or more carbocylic sugars are also suitable as nucleic acids for use in the present invention, see Jenkins et al., Chemical Society Review (1995), pages 169 to 176 as well as others which are described in Rawls, C & E News, 2 Jun. 1997, page 36, herewith incorporated by reference. Besides the selection of the nucleic acids and nucleic acid analogues known in the conventional, also any mixtures of naturally occurring nucleic acids and nucleic acid analogues or mixtures of nucleic acid analogues may be used.

In a further embodiment, the therapeutically active agent is selected from the group of metal ion complexes, as described in International Applications PCT/US95/16377, PCT/US95/16377, PCT/US96/19900, PCT/US96/15527 and herewith incorporated by reference, wherein such agents reduce or inactivate the bioactivity of their target molecules, preferable proteins such as enzymes.

Preferable therapeutically active agents are also anti-migratory, anti-proliferative or immune-suppressive, anti-inflammatory or re-endotheliating agents such as, e.g., everolimus, tacrolimus, sirolimus, mycofenolate-mofetil, rapamycin, paclitaxel, actinomycin D, angiopeptin, batimastat, estradiol, VEGF, statines and others, their derivatives and analogues.

Further preferable are active agents or combinations of active agents selected from heparin, synthetic heparin analogs (e.g., fondaparinux), hirudin, antithrombin III, drotrecogin alpha; fibrinolytics such as alteplase, plasmin, lysokinases, factor XIIa, prourokinase, urokinase, anistreplase, streptokinase; platelet aggregation inhibitors such as acetylsalicylic acid [aspirin], ticlopidine, clopidogrel, abciximab, dextrans; corticosteroids such as alclometasone, amcinonide, augmented betamethasone, beclomethasone, betamethasone, budesonide, cortisone, clobetasol, clocortolone, desonide, desoximetasone, dexamethasone, fluocinolone, fluocinonide, flurandrenolide, flunisolide, fluticasone, halcinonide, halobetasol, hydrocortisone, methylprednisolone, mometasone, prednicarbate, prednisone, prednisolone, triamcinolone; so-called non-steroidal anti-inflammatory drugs (NSAIDs) such as diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib; cytostatics such as alkaloids and podophyllum toxins such as vinblastine, vincristine; alkylating agents such as nitrosoureas, nitrogen lost analogs; cytotoxic antibiotics such as daunorubicin, doxorubicin and other anthracyclines and related substances, bleomycin, mitomycin; antimetabolites such as folic acid analogs, purine analogs or pyrimidine analogs; paclitaxel, docetaxel, sirolimus; platinum compounds such as carboplatin, cisplatin or oxaliplatin; amsacrine, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens and antiestrogens; antiarrythmics in particular class I antiarrhythmic such as antiarrhythmics of the quinidine type, quinidine, dysopyramide, ajmaline, prajmalium bitartrate, detajmium bitartrate; antiarrhythmics of the lidocaine type, e.g., lidocaine, mexiletine, phenyloin, tocainid; class Ic antiarrhythmics, e.g., propafenon, flecainid(acetate); class II antiarrhythmics beta-receptor blockers such as metoprolol, esmolol, propranolol, metoprolol, atenolol, oxprenolol; class III antiarrhythmics such as amiodarone, sotalol; class IV antiarrhythmics such as diltiazem, verapamil, gallopamil; other antiarrhythmics such as adenosine, orciprenaline, ipratropium bromide; agents for stimulating angiogenesis in the myocardium such as vascular endothelial growth factor (VEGF), basic fibroblast growth factor (bFGF), non-viral DNA, viral DNA, endothelial growth factors: FGF-1, FGF-2, VEGF, TGF; antibiotics, monoclonal antibodies, anticalins; stem cells, endothelial progenitor cells (EPC); digitalis glycosides, such as acetyl digoxin/metildigoxin, digitoxin, digoxin; cardiac glycosides such as ouabain, proscillaridin; antihypertensives such as CNS active antiadrenergic substances, e.g., methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin II antagonists: candesartancilexetil, valsartan, telmisartan, olmesartanmedoxomil, eprosartan; peripherally active alpha-receptor blockers such as prazosin, urapidil, doxazocin, bunazosine, terazosin, indoramin; vasodilators such as dihydralazine, diisopropylamine dichloroacetate, minoxidil, nitroprusside sodium; other antihypertensives such as indapamide, co-dergocrine mesylate, dihydroergotoxine methanesulfonate, cicletanin, bosentan, fludrocortisone; phosphodiesterase inhibitors such as milrinon, enoximon and antihypotensives such as in particular adrenergic and dopaminergic substances such as dobutamine, epinephrine, etilefrine, norfenefrine, norepinephrine, oxilofrine, dopamine, midodrine, pholedrine, ameziniummetil; and partial adrenoceptor agonists such as dihydroergotamine; fibronectin, polylysine, ethylene vinyl acetate, inflammatory cytokines such as: TGF, PDGF, VEGF, bFGF, TNF, NGF, GM-CSF, IGF-a, IL-1, IL 8, IL-6, growth hormone; as well as adhesive substances such as cyanoacrylates, beryllium, silica; and growth factors such as erythropoietin, hormones such as corticotropins, gonadotropins, somatropins, thyrotrophins, desmopressin, terlipressin, pxytocin, cetrorelix, corticorelin, leuprorelin, triptorelin, gonadorelin, ganirelix, buserelin, nafarelin, goserelin, as well as regulatory peptides such as somatostatin, octreotid; bone and cartilage stimulating peptides, bone morphogenetic proteins (BMPs), in particularly recombinant BMPs, such as recombinant human BMP-2 (rhBMP-2), bisphosphonate (e.g., risedronate, pamidronate, ibandronate, zoledronic acid, clodronsaure, etidronsäure, alendronic acid, tiludronic acid), fluorides such as disodium fluorophosphate, sodium fluoride; calcitonin, dihydrotachysterol; growth factors and cytokines such as epidermal growth factor (EGF), platelet-derived growth factor (PDGF), fibroblast growth factors (FGFs), transforming growth factors-b (TGFs-b), transforming growth factor-a (TGF-a), erythropoietin (EPO), insulin-like growth factor-I (IGF-I), insulin-like growth factor-II (IGF-II), interleukin-1 (IL-1), interleukin-2 (IL-2), interleukin-6 (IL-6), interleukin-8 (IL-8), tumor necrosis factor-a (TNF-a), tumor necrosis factor-b (TNF-b), interferon-g (INF-g), colony stimulating factors (CSFs); monocyte chemotactic protein, fibroblast stimulating factor 1, histamine, fibrin or fibrinogen, endothelin-1, angiotensin II, collagens, bromocriptine, methysergide, methotrexate, carbon tetrachloride, thioacetamide and ethanol; as well as silver (ions), titanium dioxide, antibiotics and anti-infective drugs such as in particular β-lactam antibiotics, e.g., β-lactamase-sensitive penicillins such as benzyl penicillins (penicillin G), phenoxymethylpenicillin (penicillin V); β-lactamase-resistant penicillins such as aminopenicillins, e.g., amoxicillin, ampicillin, bacampicillin; acylaminopenicillins such as mezlocillin, piperacillin; carboxypenicillins, cephalosporins such as cefazolin, cefuroxime, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactamase inhibitors such as sulbactam, sultamicillintosylate; tetracyclines such as doxycycline, minocycline, tetracycline, chlorotetracycline, oxytetracycline; aminoglycosides such as gentamicin, neomycin, streptomycin, tobramycin, amikacin, netilmicin, paromomycin, framycetin, spectinomycin; macrolide antibiotics such as azithromycin, clarithromycin, erythromycin, roxithromycin, spiramycin, josamycin; lincosamides such as clindamycin, lincomycin; gyrase inhibitors such as fluoroquinolones, e.g., ciprofloxacin, ofloxacin, moxifloxacin, norfloxacin, gatifloxacin, enoxacin, fleroxacin, levofloxacin; quinolones such as pipemidic acid; sulfonamides, trimethoprim, sulfadiazine, sulfalene; glycopeptide antibiotics such as vancomycin, teicoplanin; polypeptide antibiotics such as polymyxins, e.g., colistin, polymyxin-b, nitroimidazole derivates, e.g., metronidazole, tinidazole; aminoquinolones such as chloroquin, mefloquin, hydroxychloroquin; biguanids such as proguanil; quinine alkaloids and diaminopyrimidines such as pyrimethamine; amphenicols such as chloramphenicol; rifabutin, dapson, fusidic acid, fosfomycin, nifuratel, telithromycin, fusafungin, fosfomycin, pentamidine diisethionate, rifampicin, taurolidin, atovaquon, linezolid; virus static such as aciclovir, ganciclovir, famciclovir, foscarnet, inosine-(dimepranol-4-acetamidobenzoate), valganciclovir, valaciclovir, cidofovir, brivudin; antiretroviral active ingredients (nucleoside analog reverse-transcriptase inhibitors and derivatives) such as lamivudine, zalcitabine, didanosine, zidovudin, tenofovir, stavudin, abacavir; non-nucleoside analog reverse-transcriptase inhibitors: amprenavir, indinavir, saquinavir, lopinavir, ritonavir, nelfinavir; amantadine, ribavirine, zanamivir, oseltamivir or lamivudine, as well as any combinations and mixtures thereof.

In an alternative exemplary embodiment of the present invention, the active agents are encapsulated in polymers, vesicles, liposomes or micelles.

Suitable diagnostically active agents for use in the present invention can be e.g. signal generating agents or materials, which may be used as markers. Such signal generating agents include materials which in physical, chemical and/or biological measurement and verification methods lead to detectable signals, for example in image-producing methods. It is not important for the present invention, whether the signal processing is carried out exclusively for diagnostic or therapeutic purposes. Typical imaging methods are for example radiographic methods, which are based on ionizing radiation, for example conventional X-ray methods and X-ray based split image methods such as computer tomography, neutron transmission tomography, radiofrequency magnetization such as magnetic resonance tomography, further by radionuclide-based methods such as scintigraphy, Single Photon Emission Computed Tomography (SPECT), Positron Emission Computed Tomography (PET), ultrasound-based methods or fluoroscopic methods or luminescence or fluorescence based methods such as Intravasal Fluorescence Spectroscopy, Raman spectroscopy, Fluorescence Emission Spectroscopy, Electrical Impedance Spectroscopy, colorimetry, optical coherence tomography, etc, further Electron Spin Resonance (ESR), Radio Frequency (RF) and Microwave Laser and similar methods.

Signal generating agents can be metal-based from the group of metals, metal oxides, metal carbides, metal nitrides, metal oxynitrides, metal carbonitrides, metal oxycarbides, metal oxynitrides, metal oxycarbonitrides, metal hydrides, metal alkoxides, metal halides, inorganic or organic metal salts, metal polymers, metallocenes, and other organometallic compounds, chosen from powders, solutions, dispersions, suspensions, emulsions. Preferable metal based agents are especially nanomorphous nanoparticles from metals, metal oxides or mixtures there from. The metals or metal oxides used can also be magnetic; examples are—without excluding other metals—iron, cobalt, nickel, manganese or mixtures thereof, for example iron-platinum mixtures, or as an example for magnetic metal oxides, iron oxide and ferrites.

It can be preferable to use semi conducting nanoparticles, examples for this are semiconductors from group II-VI, group III-V, group IV. Group II-VI-semiconductors are for example MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe or mixtures thereof. Examples of group III-V semiconductors are for example GaAs, GaN, GaP, GaSb, InGaAs, InP, InN, InSb, InAs, AlAs, AlP, AlSb, AIS, and mixtures thereof are preferable. Germanium, lead and silicon are selected as exemplary of group IV semiconductors. The semiconductors can moreover also contain mixtures of semiconductors from more than one group, all groups mentioned above are included.

It can moreover be preferable to choose complex formed metal-based nanoparticles. Included here are so-called Core-Shell configurations, as described explicitly by Peng et al., “Epitaxial Growth of Highly Luminescent CdSe/CdS Core/Shell Nanoparticles with Photo stability and Electronic Accessibility”, Journal of the American Chemical Society, (1997) 119:7019-7029, and included herewith explicitly per reference. Preferable here are semi conducting nanoparticles, which form a core with a diameter of 1-30 nm, especially preferable of 1-15 nm, onto which other semi conducting nanoparticles crystallize in 1-50 monolayers, especially preferable are 1-15 monolayers. In this case core and shell can be present in any desired combinations as described above, in special embodiments CdSe and CdTe are preferable as the core and CdS and ZnS as the shell.

Further, signal producing metal-based agents can be selected from salts or metal ions, which preferable have paramagnetic properties, for example lead (II), bismuth (II), bismuth (III), chromium (III), manganese (II), manganese (III), iron (II), iron (III), cobalt (II), nickel (II), copper (II), praseodymium (III), neodymium (III), samarium (III), or ytterbium (III), holmium (III) or erbium (III) and the like. Based on especially pronounced magnetic moments, especially gadolinium (III), terbium (III), dysprosium (III), holmium (III) and erbium (III) are mostly preferable. Further one can select from radioisotopes. Examples of a few applicable radioisotopes include H 3, Be 10, O 15, Ca 49, Fe 60, In 111, Pb 210, Ra 220, Ra 224 and the like. Typically such ions are present as chelates or complexes, wherein for example as chelating agents or ligands for lanthanides and paramagnetic ions compounds such as diethylenetriamine pentaacetic acid (“DTPA”), ethylenediamine tetra acetic acid (“EDTA”), or tetraazacyclododecane-N,N′,N″,N′″-tetra acetic acid (“DOTA”) are used. Other typical organic complexing agents are for example published in Alexander, Chem. Rev. 95:273-342 (1995) and Jackels, Pharm. Med. Imag, Section III, Chap. 20, p645 (1990). Other usable chelating agents in the present invention, are found in U.S. Pat. Nos. 5,155,215, 5,087,440, 5,219,553, 5,188,816, 4,885,363, 5,358,704, 5,262,532, and Meyer et al., Invest. Radiol. 25: S53 (1990), further U.S. Pat. Nos. 5,188,816, 5,358,704, 4,885,363, and 5,219,553. Preferable mostly are salts and chelates from the lanthanide group with the atomic numbers 57-83 or the transition metals with the atomic numbers 21-29, or 42 or 44.

Especially preferable are paramagnetic perfluoroalkyl containing compounds which for example are described in German laid-open patents DE 196 03 033, DE 197 29 013 and in WO 97/26017, further diamagnetic perfluoroalkyl containing substances of the general formula R<PF>-L<II>-G<III>, wherein R<PF> represents a perfluoroalkyl group with 4 to 30 carbon atoms, L<II> stands for a linker and G<III> for a hydrophilic group. The linker L is a direct bond, an —SO₂— group or a straight or branched carbon chain with up to 20 carbon atoms which can be substituted with one or more —OH, —COO<->, —SO₃-groups and/or if necessary one or more —O—, —S—, —CO—, —CONH—, —NHCO—, —CONR—, —NRCO—, —SO₂—, —PO₄—, —NH—, —NR-groups, an aryl ring or contain a piperazine, wherein R stands for a C₁ to C₂₀ alkyl group, which again can contain and/or have one or a plurality of O atoms and/or be substituted with —COO<-> or SO₃— groups.

The hydrophilic group G<III> can be selected from a mono or disaccharide, one or a plurality of —COO<-> or —SO₃<->-groups, a dicarboxylic acid, an isophthalic acid, a picolinic acid, a benzenesulfonic acid, a tetrahydropyranedicarboxylic acid, a 2,6-pyridinedicarboxylic acid, a quaternary ammonium ion, an aminopolycarboxcylic acid, an aminodipolyethyleneglycol sulfonic acid, an aminopolyethyleneglycol group, an SO₂—(CH₂)₂—OH-group, a polyhydroxyalkyl chain with at least two hydroxyl groups or one or a plurality of polyethylene glycol chains having at least two glycol units, wherein the polyethylene glycol chains are terminated by an —OH or —OCH₃— group, or similar linkages. See for example published German patent DE 199 48 651, explicitly incorporated into the present invention by reference.

It can be preferable in special embodiments to choose paramagnetic metals in the form of metal complexes with phthalocyanines, especially as described in Phthalocyanine Properties and Applications, Vol. 14, C. C. Leznoff and A. B. P. Lever, VCH Ed., wherein as examples to mention are octa(1,4,7,10-tetraoxaundecyl)Gd-phthalocyanine, octa(1,4,7,10-tetraoxaundecyl)Gd-phthalocyanine, octa(1,4,7,10-tetraoxaundecyl)Mn-phthalocyanine, octa(1,4,7,10-tetraoxaundecyl)Mn-phthalocyanine, as described in U.S. 2004214810.

It can further be preferable to select from super-paramagnetic, ferromagnetic or ferrimagnetic signal generating agents. For example among magnetic metals, alloys are preferable, among ferrites such as gamma iron oxide, magnetites or cobalt-, nickel- or manganese-ferrites, corresponding agents are preferable selected, especially particles as described in International Publications WO83/03920, WO83/01738, WO85/02772 and WO89/03675, in U.S. Pat. Nos. 4,452,773, 4,675,173, and 4,770,183, and in International Publications WO88/00060, WO90/01295 and in WO90/01899.

Further, magnetic, paramagnetic, diamagnetic or super paramagnetic metal oxide crystals having diameters of less than 4000 Angstroms are especially preferable as degradable non-organic agents. Suitable metal oxides can be selected from iron oxide, cobalt oxides, iridium oxides or the like, which provide suitable signal producing properties and which have especially biocompatible properties or are biodegradable. Mostly preferable are crystalline agents of this group having diameters smaller than 500 Angstroms. These crystals can be associated covalently or non-covalently with macromolecular species and are modified such as the metal-based signal generating agents described above.

Further, zeolite containing paramagnets and gadolinium containing nanoparticles are selected from polyoxometallates, preferable of the lanthanides, (e.g., K9GdW10O36).

It is preferable to limit the average particle size of the magnetic signal producing agents to maximal 5 μm in order to optimize the image producing properties, and it is especially preferable that the magnetic signal producing particles be of a size from 2 nm up to 1 μm, most preferable 5 nm to 200 nm. The super paramagnetic signal producing agents can be chosen for example from the group of so-called SPIOs (super paramagnetic iron oxides) with a particle size larger than 50 nm or from the group of the USPIOs (ultra small super paramagnetic iron oxides) with particle sizes smaller than 50 nm.

In accordance with the present invention it can be preferable to select signal generating agents from the group of endohedral fullerenes, as disclosed for example in U.S. Pat. No. 5,688,486 or WO 9315768, which are incorporated by reference. It is further preferable to select fullerene derivatives and their metal complexes. Especially preferable are fullerene species, which comprise carbon clusters having 60, 70, 76, 78, 82, 84, 90, 96 or more carbon atoms. An overview of such species can be gathered from European patent 1331226A2 and is explicitly incorporated herein by reference.

Further metal fullerenes or endohedral carbon-carbon nanoparticles with arbitrary metal-based components can also be selected. Such endohedral fullerenes or endometallo fullerenes are particularly preferable, which for example contain rare earths such as cerium, neodymium, samarium, europium, gadolinium, terbium, dysprosium or holmium. Moreover it can be especially preferable to use carbon coated metallic nanoparticles such as carbides. The choice of nanomorphous carbon species is not limited to fullerenes, since it can be preferable to select from other nanomorphous carbon species such as nanotubes, onions, etc. In another exemplary embodiment it can be preferable to select fullerene species from non-endohedral or endohedral forms, which contain halogenated, preferable iodated, groups, as disclosed in U.S. Pat. No. 6,660,248.

In certain exemplary embodiments, mixtures of such signal generating agents of different specifications are also used, depending on the desired properties of the wanted signal generating material properties. The signal producing agents used generally can have a size of 0.5 nm to 1000 nm, preferable 0.5 nm to 900 nm, especially preferable from 0.7 to 100 nm. In this connection the metal-based nanoparticles can be provided as a powder, in polar, non-polar or amphiphilic solutions, dispersions, suspensions or emulsions. Nanoparticles are easily modifiable based on their large surface to volume ratios. The nanoparticles to be selected can for example be modified non-covalently by means of hydrophobic ligands, for example with trioctylphosphine, or be covalently modified. Examples of covalent ligands are thiol fatty acids, amino fatty acids, fatty acid alcohols, fatty acids, fatty acid ester groups or mixtures thereof, for example oleic cid and oleylamine.

In accordance with the present invention, the signal producing agents can be encapsulated in micelles or liposomes with the use of amphiphilic components, or may be encapsulated in polymeric shells, wherein the micelles/liposomes can have a diameter of 2 nm to 800 nm, preferable from 5 to 200 nm, especially preferable from 10 to 25 nm. The size of the micelles/liposomes is, without committing to a specific theory, dependant on the number of hydrophobic and hydrophilic groups, the molecular weight of the nanoparticles and the aggregation number. In aqueous solutions the use of branched or unbranched amphiphilic substances, is especially preferable in order to achieve the encapsulation of signal generating agents in liposomes/micelles. The hydrophobic nucleus of the micelles hereby contains in a exemplary embodiment a multiplicity of hydrophobic groups, preferable between 1 and 200, especially preferable between 1 and 100 and mostly preferable between 1 and 30 according to the desired setting of the micelle size.

Hydrophobic groups consist preferable of hydrocarbon groups or residues or silicon-containing residues, for example polysiloxane chains. Furthermore, they can preferable be selected from hydrocarbon-based monomers, oligomers and polymers, or from lipids or phospholipids or comprise combinations hereof, especially glyceryl esters such as phosphatidyl ethanolamine, phosphatidyl choline, or polyglycolides, polylactides, polymethacrylate, polyvinylbutylether, polystyrene, polycyclopentadienylmethylnorbornene, polyethylenepropylene, polyethylene, polyisobutylene, polysiloxane. Further for encapsulation in micelles hydrophilic polymers are also selected, especially preferable polystyrenesulfonic acid, poly-N-alkylvinylpyridiniumhalides, poly(meth)acrylic acid, polyamino acids, poly-N-vinylpyrrolidone, polyhydroxyethylmethacrylate, polyvinyl ether, polyethylene glycol, polypropylene oxide, polysaccharides such as agarose, dextrane, starches, cellulose, amylose, amylopectin, or polyethylene glycol or polyethylene imine of any desired molecular weight, depending on the desired micelles property. Further, mixtures of hydrophobic or hydrophilic polymers can be used or such lipid-polymer compositions employed. In a further special embodiment, the polymers are used as conjugated block polymers, wherein hydrophobic and also hydrophilic polymers or any desired mixtures there of can be selected as 2-, 3- or multi-block copolymers.

Such signal generating agents encapsulated in micelles can moreover be functionalized, while linker (groups) are attached at any desired position, preferable amino-, thiol, carboxyl-, hydroxyl-, succinimidyl, maleimidyl, biotin, aldehyde- or nitrilotriacetate groups, to which any desired corresponding chemically covalent or non-covalent other molecules or compositions can be bound according to the conventional. Here, especially biological molecules such as proteins, peptides, amino acids, polypeptides, lipoproteins, glycosaminoglycanes, DNA, RNA or similar bio molecules are preferable especially.

It is moreover preferable to select signal generating agents from non-metal-based signal generating agents, for example from the group of X-ray contrast agents, which can be ionic or non-ionic. Among the ionic contrast agents are included salts of 3-acetyl amino-2,4-6-triiodobenzoic acid, 3,5-diacetamido-2,4,6-triiodobenzoic acid, 2,4,6-triiodo-3,5-dipropionamido-benzoic acid, 3-acetyl amino-5-((acetyl amino)methyl)-2,4,6-triiodobenzoic acid, 3-acetyl amino-5-(acetyl methyl amino)-2,4,6-triiodobenzoic acid, 5-acetamido-2,4,6-triiodo-N-((methylcarbamoyl)methyl)-isophthalamic acid, 5-(2-methoxyacetamido)-2,4,6-triiodo-N-[2-hydroxy-1-(methylcarbamoyl)-ethoxy 1]-isophthalamic acid, 5-acetamido-2,4,6-triiodo-N-methylisophthalamic acid, 5-acetamido-2,4,6-triiodo-N-(2-hydroxyethyl)-isophthalamic acid 2-[[2,4,6-triiodo-3-[(1-oxobutyl)-amino]phenyl]methyl]-butanoic acid, beta-(3-amino-2,4,6-triiodophenyl)-alpha-ethyl-propanoic acid, 3-ethyl-3-hydroxy-2,4,6-triiodophenyl-propanoic acid, 3-[[(dimethylamino)-methyl]amino]-2,4,6-triiodophenyl-propanoic acid (see Chem. Ber. 93: 2347 (1960)), alpha-ethyl-(2,4,6-triiodo-3-(2-oxo-1-pyrrolidinyl)-phenyl)-propanoic acid, 2-[2-[3-(acetyl amino)-2,4,6-triiodophenoxy]ethoxymethyl]butanoic acid, N-(3-amino-2,4,6-triiodobenzoyl)-N-phenyl-.beta.-aminopropanoic acid, 3-acetyl-[(3-amino-2,4,6-triiodophenyl)amino]-2-methylpropanoic acid, 5-[(3-amino-2,4,6-triiodophenyl)methyl amino]-5-oxypentanoic acid, 4-[ethyl-[2,4,6-triiodo-3-(methyl amino)-phenyl]amino]-4-oxo-butanoic acid, 3,3′-oxy-bis[2,1-ethanediyloxy-(1-oxo-2,1-ethanediyl)imino]bis-2,4,6-triiodobenzoic acid, 4,7,10,13-tetraoxahexadecane-1,16-dioyl-bis(3-carboxy-2,4,6-triiodoanilide), 5,5′-(azelaoyldiimino)-bis[2,4,6-triiodo-3-(acetyl amino)methyl-benzoic acid], 5,5′-(apidoldiimino)bis(2,4,6-triiodo-N-methyl-isophthalamic acid), 5,5′-(sebacoyl-diimino)-bis(2,4,6-triiodo-N-methylisophthalamic acid), 5,5-[N,N-diacetyl-(4,9-dioxy-2,11-dihydroxy-1,12-dodecanediyl)diimino]bis(2,4,6-triiodo-N-methyl-isophthalamic acid), 5,5′5″-(nitrilo-triacetyltriimino)tris(2,4,6-triiodo-N-methyl-isophthalamic acid), 4-hydroxy-3,5-diiodo-alpha-phenylbenzenepropanoic acid, 3,5-diiodo-4-oxo-1(4H)-pyridine acetic acid, 1,4-dihydro-3,5-diiodo-1-methyl-4-oxo-2,6-pyridinedicarboxylic acid, 5-iodo-2-oxo-1(2H)-pyridine acetic acid, and N-(2-hydroxyethyl)-2,4,6-triiodo-5-[2,4,6-triiodo-3-(N-methylacetamido)-5-(methylcarbomoyl)benzamino]acetamido]-isophthalamic acid, and the like, especially preferable, as well as other ionic X-ray contrast agents suggested in the literature, for example in J. Am. Pharm. Assoc., Sci. Ed. 42:721 (1953), Swiss Patent 480071, JACS 78:3210 (1956), German patent 2229360, U.S. Pat. No. 3,476,802, Arch. Pharm. (Weinheim, Germany) 306: 11 834 (1973), J. Med. Chem. 6: 24 (1963), FR-M-6777, Pharmazie 16: 389 (1961), U.S. Pat. No. 2,705,726, U.S. Pat. No. 2,895,988, Chem. Ber. 93:2347 (1960), SA-A-68/01614, Acta Radiol. 12: 882 (1972), British Patent 870321, Rec. Trav. Chim. 87: 308 (1968), East German Patent 67209, German Patent 2050217, German Patent 2405652, Farm Ed. Sci. 28: 912 (1973), Farm Ed. Sci. 28: 996 (1973), J. Med. Chem. 9: 964 (1966), Arzheim.-Forsch 14: 451 (1964), SE-A-344166, British Patent 1346796, U.S. Pat. No. 2,551,696, U.S. Pat. No. 1,993,039, Ann 494: 284 (1932), J. Pharm. Soc. (Japan) 50: 727 (1930), and U.S. Pat. No. 4,005,188.

Examples of applicable non-ionic X-ray contrast agents in accordance with the present invention, are metrizamide as disclosed in DE-A-2031724, iopamidol as disclosed in BE-A-836355, iohexyl as disclosed in GB-A-1548594, iotrolan as disclosed in EP-A-33426, iodecimol as disclosed in EP-A-49745, iodixanol as in EP-A-108638, ioglucol as disclosed in U.S. Pat. No. 4,314,055, ioglucomide as disclosed in BE-A-846657, ioglunioe as in DE-A-2456685, iogulamide as in BE-A-882309, iomeprol as in EP-A-26281, iopentol as EP-A-105752, iopromide as in DE-A-2909439, iosarcol as in DE-A-3407473, iosimide as in DE-A-3001292, iotasul as in EP-A-22056, iovarsul as disclosed in EP-A-83964 or ioxilan in WO87/00757, and the like.

In some embodiments it is especially preferable to select agents based on nanoparticle signal generating agents, which after release into tissues and cells are incorporated or are enriched in intermediate cell compartments and/or have an especially long residence time in the organism. Such particles are selected in a special embodiment from water-insoluble agents, in another exemplary embodiment, they contain a heavy element such as iodine or barium, in a third PH-50 as monomer, oligomer or polymer (iodinated aroyloxy ester having the empirical formula C₁₉H₂₃I₃N₂O₆, and the chemical names 6-ethoxy-6-oxohexy-3,5-bis(acetyl amino)-2,4,6-triiodobenzoate), in a fourth embodiment an ester of diatrizoic acid, in a fifth an iodinated aroyloxy ester or in a sixth embodiment any combinations hereof. In these embodiments particle sizes are preferable, which can be incorporated by macrophages. A corresponding method for this is disclosed in WO03039601 and agents preferable to be selected are disclosed in the publications U.S. Pat. Nos. 5,322,679, 5,466,440, 5,518,187, 5,580,579, and 5,718,388, gel of which are explicitly incorporated by reference in accordance with the present invention. Especially advantageous are particularly, nanoparticles which are marked with signal generating agents or such signal generating agents such as PH-50, which accumulate in intercellular spaces and can make interstitial as well as extrastitial compartments visible.

Signal generating agents can be selected moreover from the group of the anionic or cationic lipids, as disclosed already in U.S. Pat. No. 6,808,720 and explicitly incorporated herewith. Especially preferable are anionic lipids such as phosphatidyl acid, phosphatidyl glycerol and their fatty acid esters, or amides of phosphatidyl ethanolamine, such as anandamide and methanandamide, phosphatidyl serine, phosphatidyl inositol and their fatty acid esters, cardiolipin, phosphatidyl ethylene glycol, acid lysolipids, palmitic acid, stearic acid, arachidonic acid, oleic acid, linoleic acid, linolenic acid, myristic acid, sulfolipids and sulfatides, free fatty acids, both saturated and unsaturated and their negatively charged derivatives, and the like. Moreover, specially halogenated, in particular fluorinated anionic lipids are preferable. The anionic lipids preferable contain cations from the alkaline earth metals beryllium (Be<+2>), magnesium (Mg<+2>), calcium (Ca<+2>), strontium (Sr<+2>) and barium (Ba<+2>), or amphoteric ions, such as aluminium (Al<+3>), gallium (Ga<+3>), germanium (Ge<+3>), tin (Sn+<4>) or lead (Pb<+2> and Pb<+4>), or transition metals such as titanium (Ti<+3> and Ti<+4>), vanadium (V<+2> and V<+3>), chromium (Cr<+2> and Cr<+3>), manganese (Mn<+2> and Mn<+3>), iron (Fe<+2> and Fe<+3>), cobalt (Co<+2> and Co<+3>), nickel (Ni<+2> and Ni<+3>), copper (Cu<+2>), zinc (Zn<+2>), zirconium (Zr<+4>), niobium (Nb<+3>), molybdenum (Mo<+2> and Mo<+3>), cadmium (Cd<+2>), indium (In <+3>), tungsten (W<+2> and W<+4>), osmium (Os<+2>, Os<+3> and Os<+4>), iridium (Ir<+2>, Ir<+3> and Ir<+4>), mercury (Hg<+2>) or bismuth (Bi<+3>), and/or rare earths such as lanthanides, for example lanthanum (La<+3>) and gadolinium (Gd<+3>). Especially preferable cations are calcium (Ca<+2>), magnesium (Mg<+2>) and zinc (Zn<+2>) and paramagnetic cations such as manganese (Mn<+2>) or gadolinium (Gd<+3>).

Cationic lipids are to be chosen from phosphatidyl ethanolamine, phospatidylcholine, Glycero-3-ethylphosphatidylcholine and their fatty acid esters, di- and tri-methylammoniumpropane, di- and tri-ethylammoniumpropane and their fatty acid esters. Especially preferable derivatives are N-[1-(2,3-dioleoyloxy)propyl]-N,N,N-trimethylammonium chloride (“DOTMA”). Furthermore synthetic cationic lipids based on for example naturally occurring lipids such as dimethyldioctadecylammonium bromide, sphingolipids, sphingomyelin, lysolipids, glycolipids such as for example gangliosides GM1, sulfatides, glycosphingolipids, cholesterol and cholesterol esters or salts, N-succinyldioleoylphosphattidyl ethanolamine, 1,2,-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidyl ethanolamine and palmitoyl-homocystein, mostly preferable are fluorinated, derivatized cationic lipids. Such compounds have been disclosed especially in U.S. Ser. No. 08/391,938.

Such lipids are furthermore suitable as components of signal generating liposomes, which especially can have pH-sensitive properties as disclosed in U.S. 2004/197392.

In accordance with the present invention, signal generating agents can also be selected from the group of the so-called microbubbles or microballoons, which contain stable dispersions or suspensions in a liquid carrier substance. Gases to be chosen are preferable air, nitrogen, carbon dioxide, hydrogen or noble gases such as helium, argon, xenon or krypton, or sulfur-containing fluorinated gases such as sulfurhexafluoride, disulfurdecafluoride or trifluoromethylsulfurpentafluoride, or for example selenium hexafluoride, or halogenated silanes such as methylsilane or dimethylsilane, further short chain hydrocarbons such as alkanes, specifically methane, ethane, propane, butane or pentane, or cycloalkanes such as cyclopropane, cyclobutane or cyclopentane, also alkenes such as ethylene, propene, propadiene or butene, or also alkynes such as acetylene or propyne. Further ethers such as dimethylether can be considered or be chosen, or ketones, or esters or halogenated short-chain hydrocarbons or any desired mixtures of the above. Especially preferable are halogenated or fluorinated hydrocarbon gases such as bromochlorodifluoromethane, chlorodifluoromethane, dichlorodifluoromethan, bromotrifluoromethane, chlorotrifluoromethane, chloropentafluoroethane, dichlorotetrafluoroethane, chlorotrifluoroethylene, fluoroethylene, ethyl fluoride, 1,1-difluoroethane or perfluorohydrocarbons such as for example perfluoroalkanes, perfluorocycloalkanes, perfluoroalkenes or perfluorinated alkynes. Especially preferable are emulsions of liquid dodecafluoropentane or decafluorobutane and sorbitol, or similar, as disclosed in WO-A-93/05819 and explicitly incorporated herewith by reference.

Preferable such micro bubbles are selected, which are encapsulated in compounds having the structure R1-X-Z; R2-X-Z; or R3-X-Z′, wherein R1, R2 comprises and R3 hydrophobic groups selected from straight chain alkylenes, alkyl ethers, alkyl thiol ethers, alkyl disulfides, polyfluoroalkylenes and polyfluoroalkylethers, Z comprises a polar group from CO₂-M<+>, SO₃<->M<+>, SO4<->M<+>, PO₃<->M<+>, PO₄<-> M<+2>, N(R)₄<+> or a pyridine or substituted pyridine, and a zwitterionic group, M is a metal ion, and finally X represents a linker which binds the polar group with the residues.

Gas-filled or in situ out-gassing micro spheres having a size of less than 1000 μm can be further selected from biocompatible synthetic polymers or copolymers which comprise monomers, dimers or oligomers or other pre-polymer to pre-stages of the following polymerizable substances: acrylic acid, methacrylic acid, ethyleneimine, crotonic acid, acryl amide, ethyl acrylate, methylmethacrylate, 2-hydroxyethylmethacrylate (HEMA), lactonic acid, glycolic acid, [epsilon]caprolactone, acrolein, cyanoacrylate, bisphenol A, epichlorhydrin, hydroxyalkylacrylate, siloxane, dimethylsiloxane, ethylene oxide, ethylene glycol, hydroxyalkylmethacrylate, N-substituted acryl amide, N-substituted methacrylamides, N-vinyl-2-pyrrolidone, 2,4-pentadiene-1-ol, vinyl acetate, acrylonitrile, styrene, p-aminostyrene, p-aminobenzylstyrene, sodium styrenesulfonate, sodium-2-sulfoxyethylmethacrylate, vinyl pyridine, aminoethylmethacrylate, 2-methacryloyloxytrimethylammonium chloride, and polyvinylidenes, such as polyfunctional cross-linkable monomers such as for example N,N′-methylene-bis-acrylamide, ethylene glycol dimethacrylate, 2,2′-(p-phenylenedioxy)-diethyldimethacrylate, divinylbenzene, triallylamine and methylene-bis-(4-phenyl-isocyanate), including any desired combinations thereof. Preferable polymers contain polyacrylic acid, polyethyleneimine, polymethacrylic acid, polymethylmethacrylate, polysiloxane, polydimethylsiloxane, polylactonic acid, poly([epsilon]-caprolactone), epoxy resins, poly(ethylene oxide), poly(ethylene glycol), and polyamides (e.g. Nylon) and the like or any arbitrary mixtures thereof. Preferable copolymers contain among others polyvinylidene-polyacrylonitrile, polyvinylidene-polyacrylonitrile-polymethylmethacrylate, and polystyrene-polyacrylonitrile and the like or any desired mixtures thereof. Methods for manufacture of such micro spheres are published for example in Garner et al., U.S. Pat. No. 4,179,546, Garner, U.S. Pat. No. 3,945,956, Cohrs et al., U.S. Pat. No. 4,108,806, Japan Kokai Tokkyo Koho 62 286534, British Patent 1,044,680, Kenaga et al., U.S. Pat. No. 3,293,114, Morehouse et al., U.S. Pat. No. 3,401,475, Walters, U.S. Pat. No. 3,479,811, Walters et al., U.S. Pat. No. 3,488,714, Morehouse et al., U.S. Pat. No. 3,615,972, Baker et al., U.S. Pat. No. 4,549,892, Sands et al., U.S. Pat. No. 4,540,629, Sands et al., U.S. Pat. No. 4,421,562, Sands, U.S. Pat. No. 4,420,442, Mathiowitz et al., U.S. Pat. No. 4,898,734, Lencki et al., U.S. Pat. No. 4,822,534, Herbig et al., U.S. Pat. No. 3,732,172, Himmel et al., U.S. Pat. No. 3,594,326, Sommerville et al., U.S. Pat. No. 3,015,128, Deasy, Microencapsulation and Related Drug Processes, Vol. 20, Chapters. 9 and 10, pp. 195-240 (Marcel Dekker, Inc., N.Y., 1984), Chang et al., Canadian J of Physiology and Pharmacology, Vol 44, pp. 115-129 (1966), and Chang, Science, Vol. 146, pp. 524-525 (1964).

Other signal generating agents can in accordance with the present invention be selected from the group of agents, which are transformed into signal generating agents in organisms by means of in-vitro or in-vivo cells, cells as a component of cell cultures, of in-vitro tissues, or cells as a component of multicellular organisms, such as for example fungi, plants or animals, in exemplary embodiments from mammals such as mice or humans. Such agents can be made available in the form of vectors for the transfection of multicellular organisms, wherein the vectors contain recombinant nucleic acids for the coding of signal generating agents. In certain exemplary embodiments this is concerned with signal generating agents such as metal binding proteins. It can be preferable to choose such vectors from the group of viruses for example from adeno viruses, adeno virus associated viruses, herpes simplex viruses, retroviruses, alpha viruses, pox viruses, arena-viruses, vaccinia viruses, influenza viruses, polio viruses or hybrids of any of the above.

Further such signal generating agents are to be chosen in combination with delivery systems, in order to incorporate nucleic acids, which are suitable for coding for signal generating agents, into the target structure. Especially preferable are virus particles for the transfection of mammalian cells, wherein the virus particle contains one or a plurality of coding sequence/s for one or a plurality of signal generating agents as described above. In these cases the particles are generated from one or a plurality of the following viruses: adeno viruses, adeno virus associated viruses, herpes simplex viruses, retroviruses, alpha viruses, pox viruses, arena-viruses, vaccinia viruses, influenza viruses and polio viruses.

In further embodiments, these signal generating agents are made available from colloidal suspensions or emulsions, which are suitable to transfect cells, preferable mammalian cells, wherein these colloidal suspensions and emulsions contain those nucleic acids which possess one or a plurality of the coding sequence(s) for signal generating agents. Such colloidal suspensions or emulsions can contain macromolecular complexes, nano capsules, microspheres, beads, micelles, oil-in-water- or water-in-oil emulsions, mixed micelles and liposomes or any desired mixture of the above.

In further embodiments, cells, cell cultures, organized cell cultures, tissues, organs of desired species and non-human organisms can be chosen which contain recombinant nucleic acids having coding sequences for signal generating agents. In certain exemplary embodiments organisms are selected from the groups: mouse, rat, dog, monkey, pig, fruit fly, nematode worms, fish or plants or fungi. Further, cells, cell cultures, organized cell cultures, tissues, organs of desired species and non-human organisms can contain one or a plurality of vectors as described above.

Signal generating agents are preferable produced in vivo from the group of proteins and made available as described above. Such agents are preferable directly or indirectly signal producing, while the cells produce (direct) a signal producing protein through transfection or produce a protein which induces (indirect) the production of a signal producing protein. Preferable these signal generating agents are detectable in methods such as MRI while the relaxation times T1, T2 or both are altered and lead to signal producing effects which can be processed sufficiently for imaging. Such proteins are preferable protein complexes, especially metalloprotein complexes. Direct signal producing proteins are preferable such metalloprotein complexes which are formed in the cells. Indirect signal producing agents are such proteins or nucleic acids, for example, which regulate the homeostasis of iron metabolism, the expression of endogenous genes for the production of signal generating agents, and/or the activity of endogenous proteins with direct signal generating properties, for example Iron Regulatory Protein (IRP), Transferrin receptor (for the take-up of Fe), erythroid-5-aminobevulinate synthase (for the utilization of Fe, H-Ferritin and L-Ferritin for the purpose of Fe storage). In certain exemplary embodiments it can be preferable to combine both types of signal generating agents, that is direct and indirect, with each other, for example an indirect signal generating agent, which regulates the iron-homeostasis and a direct agent, which represents a metal binding protein.

In such embodiments, where preferable metal-binding polypeptides are selected as indirect agents, it is advantageous if the polypeptide binds to one or a plurality of metals which possess signal generating properties. Especially preferable are such metals with unpaired electrons in the Dorf orbitals, such as for example Fe, Co, Mn, Ni, Gd etc., wherein especially Fe is available in high physiological concentrations in organisms. It is moreover preferable, if such agents form metal-rich aggregates, for example crystalline aggregates, whose diameters are larger than 10 picometers, preferable larger than 100 picometers, 1 nm, 10 nm or specially preferable larger than 100 nm.

Preferable are such metal-binding compounds, which have sub-nanomolar affinities with dissociation constants of less than 10⁻¹⁵ M, 10⁻² M or smaller. Typical polypeptides or metal-binding proteins are lactoferrin, ferritin, or other dimetallocarboxylate proteins or the like, or so-called metal catcher with siderophoric groups, such as for example haemoglobin. A possible method for preparation of such signal generating agents, their selection and the possible direct or indirect agents which are producible in vivo and are suitable as signal generating agents was disclosed in WO 03/075747 and is incorporated herewith in accordance with the present invention.

Another group of signal generating agents can be photophysically signal producing agents which consist of dyestuff-peptide-conjugates. Such dyestuff-peptide-conjugates are preferable which provide a wide spectrum of absorption maxima, for example polymethin dyestuffs, in particular cyanine-, merocyanine-, oxonol- and squarilium dyestuffs. From the class of the polymethin dyestuffs the cyanine dyestuffs, e.g. the indole structure based indocarbo-, indodicarbo- and indotricarbocyanines, are especially preferable. Such dyestuffs can be preferable in certain exemplary embodiments, which are substituted with suitable linking agents and can be functionalized with other groups as desired. In this connection see also DE 19917713.

In accordance with the present invention, signal generating agents can be functionalized as desired. The functionalization by means of so-called “Targeting” groups is preferable are to be understood, as functional chemical compounds which link the signal generating agent or its specifically available form (encapsulation, micelles, micro spheres, vectors etc.) to a specific functional location, or to a determined cell type, tissue type or other desired target structures. Preferable targeting groups permit the accumulation of signal-producing agents in or at specific target structures. Therefore the targeting groups can be selected from such substances, which are principally suitable to provide a purposeful enrichment of the signal generating agents in their specifically available form by physical, chemical or biological routes or combinations thereof. Useful targeting groups to be selected can therefore be antibodies, cell receptor ligands, hormones, lipids, sugars, dextrane, alcohols, bile acids, fatty acids, amino acids, peptides and nucleic acids, which can be chemically or physically attached to signal-generating agents, in order to link the signal-generating agents into/onto a specifically desired structure. In a first embodiment targeting groups are selected, which enrich signal-generating agents in/on a tissue type or on surfaces of cells. Here it is not necessary for the function, that the signal generating agent be taken up into the cytoplasm of the cells. Peptides are preferable as targeting groups, for example chemotactic peptides are used to make inflammation reactions in tissues visible by means of signal generating agents; in this connection see also WO 97/14443.

Antibodies are also preferable, including antibody fragments, Fab, Fab2, Single Chain Antibodies (for example Fv), chimerical antibodies, and the like, as known from the conventional, moreover antibody-like substances, for example so-called anticalines, wherein it is unimportant whether the antibodies are modified after preparation, recombinants are produced or whether they are human or non-human antibodies. It is preferable to choose from humanized or human antibodies, examples of humanized forms of non-human antibodies are chimerical immunoglobulines, immunoglobulin chains or fragments (such as Fv, Fab, Fab′, F(ab″)₂ or other antigen-binding subsequences of antibodies, which partly contain sequences of non-human antibodies; humanized antibodies contain for example human immunoglobulines (receptor or recipient antibody), in which groups of a CDR (Complementary Determining Region) of the receptor are replaced through groups of a CDR of a non-human (spender or donor antibody), wherein the spender species for example, mouse, rabbit or other has appropriate specificity, affinity, and capacity for the binding of target antigens. In a few forms the Fv framework groups of the human immunglobulines are replaced by means of corresponding non-human groups. Humanized antibodies can moreover contain groups which either do not occur in either the CDR or Fv framework sequence of the spender or the recipient. Humanized antibodies essentially comprise substantially at least one or preferable two variable domains, in which all or substantial components of the CDR components of the CDR regions or Fv framework sequences correspond with those of the non-human immunoglobulin, and all or substantial components of the FR regions correspond with a human consensus-sequence. In accordance with the present invention targeting groups of this embodiment can also be hetero-conjugated antibodies. Preferable function of the selected antibodies or peptides are cell surface markers or molecules, particularly of cancer cells, wherein here a large number of known surface structures are known, such as HER2, VEGF, CA15-3, CA 549, CA 27.29, CA 19, CA 50, CA242, MCA, CA125, DE-PAN-2, etc., and the like.

Moreover, it is preferable to select targeting groups which contain the functional binding sites of ligands. Such can be chosen from all types, which are suitable for binding to any desired cell receptors. Examples of possible target receptors are, without limiting the choice, receptors of the group of insulin receptors, insulin-like growth factor receptor (e IGF-1 and IGF-2), growth hormone receptor, glucose transporters (particularly GLUT 4 receptor), transferrin receptor (transferrin), Epidermal Growth Factor receptor (EGF), low density lipoprotein receptor, high density lipoprotein receptor, leptin receptor, oestrogen receptor; interleukin receptors including IL-1, IL-2, IL-3, IL-4, IL-5, IL-6, IL-7, IL-8, IL-9, IL-11, IL-12, IL-13, IL-15, and IL-17 receptor, VEGF receptor (VEGF), PDGF receptor (PDGF), Transforming Growth Factor receptor (including TGF-[alpha] and TGF-[beta]), EPO receptor (EPO), TPO receptor (TPO), ciliary neurotrophic factor receptor, prolactin receptor, and T-cell receptors.

It can be preferable to select hormone receptors, especially for hormones such as steroidal hormones or protein- or peptide-based hormones, for example, however not limited thereto, epinephrines, thyroxines, oxytocine, insulin, thyroid-stimulating hormone, calcitonine, chorionic gonadotropine, corticotropine, follicle stimulating hormone, glucagons, leuteinizing hormone, lipotropine, melanocyte-stimulating hormone, norepinephrines, parathyroid hormone, Thyroid-Stimulating Hormone (TSH), vasopressin's, encephalin, serotonin, estradiol, progesterone, testosterone, cortisone, and glucocorticoide. Receptor ligands include those which are on the cell surface receptors of hormones, lipids, proteins, glycol proteins, signal transducers, growth factors, cytokine, and other bio molecules. Moreover, targeting groups can be selected from carbohydrates with the general formula: Cₓ(H₂O)_{y}, wherein herewith also monosaccharides, disaccharides and oligo—as well as polysaccharides are included, as well as other polymers which consist of sugar molecules which contain glycosidic bonds. Specially preferable carbohydrates are those in which all or parts of the carbohydrate components contain glycosylated proteins, including the monomers and oligomers of galactose, mannose, fructose, galactosamine, glucosamine, glucose, sialic acid, and especially the glycosylated components, which make possible the binding to specific receptors, especially cell surface receptors. Other useful carbohydrates to be selected contain monomers and polymers of glucose, ribose, lactose, raffinose, fructose and other biologically occurring carbohydrates especially polysaccharides, for example, however not exclusively, arabinogalactan, gum Arabica, mannan and the like, which are usable in order to introduce signal generating agents into cells. Reference is made in this connection to U.S. Pat. No. 5,554,386.

Furthermore targeting groups can be selected from the lipid group, wherein also fats, fatty oils, waxes, phospholipids, glycolipids, terpenes, fatty acids and glycerides, especially triglycerides are included. Further included are eicosanoides, steroids, sterols, suitable compounds of which can also be hormones such as prostaglandins, opiates and cholesterol and the like. In accordance with the present invention, all functional groups can be selected as the targeting group, which possess inhibiting properties, such as for example enzyme inhibitors, preferable those which link signal generating agents into/onto enzymes.

In a second embodiment, targeting groups can be selected from a group of functional compounds which make possible internalization or incorporation of signal generating agents in the cells, especially in the cytoplasm or in specific cell compartments or organelles, such as for example the cell nucleus. For example targeting group is preferable which contains all or parts of HIV-1 tat-proteins, their analogs and derivatized or functionally similar proteins, and in this way allows an especially rapid uptake of substances into the cells. As an example refer to Fawell et al., PNAS USA 91:664 (1994); Frankel et al., Cell 55:1189, (1988); Savion et al., J. Biol. Chem. 256:1149 (1981); Derossi et al., J. Biol. Chem. 269:10444 (1994); and Baldin et al., EMBO J. 9:1511 (1990).

Targeting groups can be further selected from the so-called Nuclear Localisation Signal (NLS), where under short positively charged (basic) domains are understood which bind to specifically targeted structures of cell nuclei. Numerous NLS and their amino acid sequences are known including single basic NLS such as that of the SV40 (monkey virus) large T Antigen (pro Lys Lys Lys Arg Lys Val), Kalderon (1984), et al., Cell, 39:499-509), the teinoic acid receptor-[beta] nuclear localization signal (ARRRRP); NFKB p50 (EEVQRKRQKL; Ghosh et al., Cell 62:1019 (1990); NFKB p65 (EEKRKRTYE; Nolan et al., Cell 64:961 (1991), as well as others (see for example Boulikas, J. Cell. Biochem. 55(1):32-58 (1994), and double basic NLS's such as for example xenopus (African clawed toad) proteins, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849, 1988. These are all incorporated herewith by reference in accordance with the present invention. Numerous localization studies have shown that NLSs, which are built into synthetic peptides which normally do not address the cell nucleus or were coupled to reporter proteins, lead to an enrichment of such proteins and peptides in cell nuclei. In this connection exemplary references are made to Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990. It can be especially preferable to select targeting groups for the hepatobiliary system, wherein in U.S. Pat. Nos. 5,573,752 and 5,582,814 corresponding groups are suggested.

In some embodiments, the implant comprises absorptive agents, e.g. to remove compounds from body fluids. Suitable absorptive agents, but not exclusively and not limited to, are chelating agents such as penicillamine, methylene tetramine dihydrochloride, EDTA, DMSA or deferoxamine mesylate, any other appropriate chemical modification of the coating surface, antibodies, and microbeads or other materials containing cross linked reagents for absorption of drugs, toxins or other agents.

In some specifically exemplary embodiments biologically active agents are selected from cells, cell cultures, organized cell cultures, tissues, organs of desired species and non-human organisms.

In certain exemplary embodiments, the beneficial agents comprise metal based nano-particles that are selected from ferromagnetic or superparamagnetic metals or metal-alloys, either further modified by coating with silanes or any other suitable polymer or not modified, for interstitial hyperthermia or thermoablation. In further embodiments, the exemplary implants can comprise silver nano-particles or other anti-infective inorganic materials, preferable as nano-particles with a D50 between 10 nm and 50 nm, whereby the amount of the anti-infective particles is at least 1 weight %, preferable 2-5 weight % and more preferable 5 to 10 weight %, most preferable between 10 and 20 weight %.

In another exemplary embodiment it can be desirable to coat the implant on the outer surface or inner surface with a coating to enhance engraftment or biocompatibility. Such coatings may comprise carbon coatings, metal carbides, metal nitrides, metal oxides e.g. diamond-like carbon or silicon carbide, or pure metal layers of e.g. titanium, using PVD, Sputter-, CVD or similar vapor deposition methods or ion implantation.

In further embodiments it can be preferable to produce a porous coating onto at least one part of the exemplary implant in a further step, such as porous carbon coatings as disclosed in WO 2004/101177, WO 2004/101017 or WO 2004/105826, or porous composite-coatings as disclosed previously in PCT/EP2006/063450, or porous metal-based coatings as disclosed in WO 2006/097503, or any other suitable porous coating.

In further embodiments a sol/gel-based beneficial agent can be incorporated into the exemplary implant or a sol/gel-based coating that can be dissolvable in physiologic fluids may be applied to at least a part of the implant, as disclosed e.g. in WO 2006/077256 or WO 2006/082221.

In some exemplary embodiments it can be desirable to combine two or more different functional modifications as described above to obtain a functional implant.

It should be noted that the term ‘comprising’ does not exclude other elements or steps and the ‘a’ or ‘an’ does not exclude a plurality. In addition elements described in association with the different embodiments may be combined.

It should be noted that the reference signs in the claims shall not be construed as limiting the scope of the claims.

Having thus described in detail several exemplary embodiments of the present invention, it is to be understood that the present invention described above is not to be limited to particular details set forth in the above description, as many apparent variations thereof are possible without departing from the spirit or scope of the present invention. The exemplary embodiments of the present invention are disclosed herein or are obvious from and encompassed by the detailed description. The detailed description, given by way of example, but not intended to limit the present invention solely to the specific embodiments described, may best be understood in conjunction with the accompanying Figures.

The foregoing applications, and all documents cited therein or during their prosecution (“appln. cited documents”) and all documents cited or referenced in the appln. cited documents, and all documents cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference, and may be employed in the practice of the present invention.

What is claimed is:

1. An at least partially biodegradable implant suitable for implantation into a subject for repairing a bone or cartilage defect, comprising:
a three-dimensional open-celled framework structure composed of a first non-particulate first material, the framework structure being embedded in a second non-particulate material different from the first material, or the open-celled framework structure being substantially completely filled with said second, non-particulate material, wherein at least one of the first material or the second material is at least partially degradable in-vivo.

2. The implant of claim 1, wherein the implant is substantially non-porous.

3. The implant of claim 1, wherein the unfilled or not embedded framework structure made of a non-particulate first material has a bulk volume porosity of about 10-90%.

4. The implant of claim 3, wherein the unfilled or not embedded framework structure has the form of a spongy or trabecular open-spaced lattice including, interconnected channels or interconnected pores.

5. The implant of claim 4, wherein the channels and/or pores have a dimension suitable for osteoconduction of about 200 to 1000 μm.

6. The implant of claim 1, wherein the first material or the second material includes at least one of a metal or a metal alloy.

7. The implant of claim 1, wherein at least one of the first material or the second material is completely degradable in-vivo.

8. The implant of claim 7, wherein at least one of the degradable first or second material includes at least one metal selected from an alkaline metal, an alkaline earth metal, Fe, Zn, Al, Mg, Ca, Zn, W, Ln, Si, or Y.

9. The implant of claim 7, wherein the degradable first or second material is combined with at least one other metal selected from at least one of Mn, Co, Ni, Cr, Cu, Cd, Pb, Sn, Th, Zr, Ag, Au, Pd, Pt, Si, Ca, Li, Al, Zn or Fe.

10. The implant of claim 5, wherein the first material or the second material includes a magnesium alloy comprising more than 90% of Mg, about 4-5% of Y, and about 1.5-4% of other rare earth metals.

11. The implant of claim 6, wherein the degradable non-particulate metallic material comprises a metal alloy of:
(i) 10-98 wt.-%, such as 35-75 wt.-% of Mg, and 0-70 wt.-%, such as 30-40% of Li and 0-12 wt.-% of other metals, or
(ii) 60-99 wt.-% of Fe, 0.05-6 wt.-% Cr, 0.05-7 wt.-% Ni and up to 10 wt.-% of other metals; or
(iii) 60-96 wt.-% Fe, 1-10 wt.-% Cr, 0.05-3 wt.-% Ni and 0-15 wt.-% of other metals, wherein the individual weight ranges are selected to add up to 100 wt.-% in total for each alloy.

12. The implant of any one of claims 1 to 6, wherein one of the non-particulate first or second material is substantially not degradable in-vivo.

13. The implant of claim 11, wherein the first or second material includes at least one metal selected from the group of main group metals of the periodic system, transition metals such as copper, gold and silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or from rare earth metals.

14. The implant of claim 6, wherein the first or second material includes a biocorrosive alloy such as biocorrosive alloys comprising as a major component tungsten, rhenium, osmium or molybdenum.

15. The implant of claim 14, wherein the biocorrosive alloy further comprises cerium, an actinide, iron, tantalum, platinum, gold, gadolinium, yttrium or scandium.

16. The implant of claim 6, wherein the non-particulate first or second material comprises a mixture of at least one first metallic material and at least one second metallic material, the first metallic material being more electronegative than the second metallic material, such that the first and second non-particulate metallic material form a local cell at their contact surfaces.

17. The implant of claim 1, wherein at least one of the first material or the second material is an organic material.

18. The implant of claim 17, wherein the organic material comprises an oligomer, polymer or copolymer selected from at least one of a poly(meth)acrylate, unsaturated polyester, saturated polyester, polyolefines, polyethylene, polypropylene, polybutylene, alkyd resins, epoxy-polymers or resins, polyamide, polyimide, polyetherimide, polyamideimide, polyesterimide, polyester amide imide, polyurethane, polycarboxylate, polycarbonate, polystyrene, polyphenol, polyvinyl ester, polysilicone, polyacetal, cellulosic acetate, polyvinylchloride, polyvinyl acetate, polyvinyl alcohol, polysulfone, polyphenylsulfone, polyethersulfone, polyketone, polyetherketone, polybenzimidazole, polybenzoxazole, polybenzthiazole, polyfluorocarbons, polyphenylene ether, polyarylate, or cyanatoester-polymers, and any of the copolymers and any mixtures thereof.

19. The implant of claim 17, wherein the organic material comprises a polymer or copolymer selected from at least one of collagen, albumin, gelatin, hyaluronic acid, starch, cellulose, methylcellulose, hydroxypropylcellulose, hydroxypropylmethyl-cellulose, carboxymethylcellulose-phthalate; gelatin, casein, dextrane, polysaccharide, fibrinogen, poly(D,L lactide), poly(D,L-lactide-co-glycolide), poly(glycolide-co-trimethylene carbonates), poly(glycolide), poly(hydroxybutylate), poly(allylcarbonate), poly(a-hydroxyesters), poly(ether esters, poly(orthoester), polyester, poly(hydroxyvaleric acid), polydioxanone, poly(ethylene terephthalate), poly(maleic acid), poly(malic acid), poly(tartaric acid), polyanhydride, polyphosphazene, poly(amino acids), polypeptides, polycaprolactones, poly(propylene fumarates), poly(ester amides), poly(ethylene fumarates), poly(hydroxy butyrates), and polyurethanes.

20. The implant of claim 17, wherein the organic material is at least partially biodegradable in-vivo.

21. The implant of claims 1, wherein the first material or the second material includes an inorganic-organic hybrid material, obtainable by sol-gel processing.

22. The implant of claim 1, wherein the non-particulate first material includes a metal, a metal alloy, or a ceramic material, and the second material includes an organic material or an inorganic-organic hybrid material, obtainable by sol-gel processing.

23. The implant of claim 1, wherein the non-particulate first material includes an organic material or an inorganic-organic hybrid material, obtainable by sol-gel processing, and the second material includes a metal, a metal alloy, or a ceramic material.

24. The implant of claim 1, wherein the non-particulate first material includes a metal or a metal alloy, and the second material includes an organic material, or wherein the non-particulate first material includes an organic material, and the second material includes a metal or a metal alloy.

25. The implant of claim 1, further comprising, in at least one of the first material or the second material, at least one additive such as an inorganic or organic filler, preferable an inorganic filler such as silica powder, silver nanoparticles, quartz, glass beads, aluminum oxide, ceramics, salts, hydroxyl apatite; a pigment; or a beneficial agent.

26. The implant of claim 25, wherein the beneficial agent includes at least one of a pharmacologically, therapeutically, biologically or diagnostically active agent or an absorptive agent.

27. The implant of claim 26, wherein the beneficial ingredient is configured to be released in-vivo from the final implant.

28. The implant of claim 1, wherein the first material comprises at least 5 wt.-% of the implant.

29. The implant of claim 1, wherein the second material comprises at least 5 wt.-% of the implant.

30. The implant of claim 1, further comprising a Youngs modulus corresponding to cancellous natural bone in the range from about 0.01 to about 2 GPa.

31. The implant of claim 1, further comprising a Youngs modulus corresponding to cortical natural bone in the range from about 15 to about 30 GPa.

32. The implant of claim 1, wherein the second material is substantially non-degradable in-vivo.

33. The implant of claim 1, wherein the first material and the second material is degradable in-vivo.

34. The implant of claim 33, wherein the in-vivo degradation rate of the first material and the second material is different.

35. The implant of claim 34, wherein the in-vivo degradation rate of the second material is lower than the degradation rate of the first material.

36. The implant of claim 34, wherein the in-vivo degradation rate of the second material is higher than the degradation rate of the first material.

37. The implant of claim 32, wherein the first material or the second material is selected such that its in-vivo degradation rate matches with the re-growth or repair rate of the natural bone, wherein the degradation rate of the material is preferable in a range of from about 3 to 8 weeks.

38. The implant of claim 32, wherein the first material or the second material is selected such that its in-vivo degradation rate matches with the regrowth or repair rate of the natural cartilage, wherein the degradation rate is preferable in a range of from about 4 to 10 weeks.

39. The implant of claim 1, wherein the implant is selected from one of a bone tissue or cartilage replacement, an implantable fracture fixation device such as plates, screws and rods, a dental implant, an orthopedic implant, a traumatologic implant, or a surgical implant.

40. A method for repairing a bone or cartilage defect in a living organism, comprising implanting an implant into the defective bone or cartilage or replacing the defective bone or cartilage at least partially, wherein the implant is at least partially biodegradable implant suitable provided for implantation into a subject for repairing a bone or cartilage defect, the implant comprising:
a three-dimensional open-celled framework structure composed of a first non-particulate first material, the framework structure being embedded in a second non-particulate material different from the first material, or the open-celled framework structure being substantially completely filled with said second, non-particulate material, wherein at least one of the first material or the second material is at least partially degradable in-vivo.

41. The method of claim 40, wherein the defect includes a defect or wound in a bone, tooth or cartilage of a living organism.

42. A utilization of an implant of for repairing a bone, tooth or cartilage defect in a living organism, wherein the implant is at least partially biodegradable implant suitable provided for implantation into a subject for repairing a bone or cartilage defect, the implant comprising:
a three-dimensional open-celled framework structure composed of a first non-particulate first material, the framework structure being embedded in a second non-particulate material different from the first material, or the open-celled framework structure being substantially completely filled with said second, non-particulate material, wherein at least one of the first material or the second material is at least partially degradable in-vivo.
